(19) United States
(12) Patent Application Publication
(10) Pub. No.: US 2023/0414872 A1
(43) Pub. Date: Dec. 28, 2023

(54) DRUG INJECTION DEVICE BASED ON ELECTROCHEMICAL REACTION AND FABRICATION METHOD FOR DRUG INJECTION PUMP

(71) Applicant: PEKING UNIVERSITY, Beijing (CN)

(72) Inventor: Yue CUI, Beijing (CN)

(21) Appl. No.: 18/339,332

(22) Filed: Jun. 22, 2023

(30) Foreign Application Priority Data
Jun. 24, 2022 (CN) 202210724624.2
Jun. 24, 2022 (CN) 202221609082.6
Jun. 24, 2022 (CN) 202221611164.4

(51) Int. Cl. A61M 5/172; A61M 5/142

(52) U.S. Cl. CPC A61M 5/1723; A61M 5/142; A61M 2230/201; A61M 2005/14208; A61M 2205/3303

(57) ABSTRACT

A drug injection device based on an electrochemical reaction and a fabrication method for a drug injection pump, belonging to the technical field of medical devices are provided. A driving force is generated based on electrochemical reaction, and drug solution is automatically driven by the driving force to administer a drug to a patient. Meanwhile, three specific structures of the drug injection device based on the electrochemical reaction are provided, namely, a first drug injection pump based on electrochemical reaction, an insulin injection system, and a closed-loop control system, which can achieve the automation of the drug administration process.

### CROSS-REFERENCE TO RELATED APPLICATION

This application claims the priority of Chinese Patent Application No. 202210724624.2 field with the China National Intellectual Property Administration on Jun. 24, 2022 and entitled with “Drug injection pump based on electrochemical reaction and fabrication method thereof”, Chinese Patent Application No. 202221609082.6 field with the China National Intellectual Property Administration on Jun. 24, 2022 and entitled with “Closed-loop control system and closed-loop system for insulin injection”, and Chinese Patent Application No. 202221611164.4 field with the China National Intellectual Property Administration on Jun. 24, 2022 and entitled with “Insulin injection system”, the disclosures of which are incorporated herein in their entirety by reference.

### TECHNICAL FIELD

The present disclosure belongs to the technical field of medical devices, and in particular to a drug injection device based on an electrochemical reaction, and a fabrication method for a drug injection pump.

### BACKGROUND

In the medical field, some patients need frequent injections of corresponding drugs for a long time for special reasons, so as to meet the requirements of maintaining the stability and health of the corresponding functions of the body, such as continuous analgesia for surgical patients or pain-suffered patients, or insulin supplementation for diabetics, and the administration requirement is continuous and stable. At present, manual injection for administration is carried out by medical staff or patients themselves at certain time intervals. However, the stability of administration cannot be guaranteed as manual operation is difficult to strictly observe the time, and the actual needs of different individuals cannot be satisfied as the administration time and drug injection amount cannot be adjusted in manual operation, and thus the intelligence and automation of drug administration cannot be achieved.

### SUMMARY

An objective of the present disclosure is to provide a drug injection device based on an electrochemical reaction, and a fabrication method for a drug injection pump, so as to solve the problem of automation in the drug administration process.

In order to achieve the above objective, the embodiments of the present disclosure provide the following solution:

A drug injection device based on an electrochemical reaction is provided. The drug injection device based on the electrochemical reaction is used to generate a driving force based on an electrochemical reaction, and to automatically drive drug solution under the driving force to administer a drug to a patient.

The drug injection device based on the electrochemical reaction provided by the embodiment is based on the electrochemical reaction and achieves the automation of the drug administration process.

Further, the present disclosure also provides three specific structures of above drug injection device based on the electrochemical reaction.

First, the drug injection device based on the electrochemical reaction is a first drug injection pump based on an electrochemical reaction, and the first drug injection pump includes a driving component and a drug storage component.

The driving component is arranged inside the drug storage component, and is used to generate a driving force based on an electrochemical reaction principle. The driving force is applied inside the drug storage component. The driving component includes an electrochemical element which is connected to the outside of the drug storage component via a wire and is used for receiving a preset current. The electrochemical element is used for generating a gas based on the preset current, and the gas is used for generating the driving force, and the electrochemical element is an electrode with a nano or micron thickness fabricated by a metal evaporation process, a screen-printing process or a magnetron sputtering process.

The drug storage component is internally loaded with drug solution, and the drug solution is pushed to the outside of the drug storage component along at least one liquid outlet hole on the drug storage component under the driving force, thus administering a drug to a patient through the liquid outlet hole, or administering the drug solution to a patient along an injection mechanism connected to the liquid outlet hole.

As can be seen from the first technical solution, the drug injection pump includes a driving component and a drug storage component. The driving component is located inside the drug storage component and is used to generate a driving force inside the drug storage component by utilizing a current based on an electrochemical principle. The drug storage component is internally loaded with drug solution, which is used to push out the drug solution and inject the drug solution into a patient under the push of the driving component. As the electrochemical reaction may be generated based on a preset regular electrical signal, the patient can be injected on time by controlling an input current or voltage, thus ensuring the stability, intelligence and automation of drug administration.

Second: the drug injection device based on the electrochemical reaction is a closed-loop control insulin injection system based on a microtube sensor. The closed-loop control insulin injection system includes a second drug injection pump based on an electrochemical principle, a first sensor, a sensor circuit module, a pump drive circuit module, and a controller.

The first sensor is attached to the skin of a patient and is used for generating a current signal based on glucose in subcutaneous tissue fluid.

The sensor circuit module is connected to the sensor and is used for receiving the current signal and outputting a glucose concentration matched with the current signal via an output end of the sensor circuit module.

The controller is provided with a signal input end and a signal output end. The signal input end is connected to the output end of the sensor circuit module and is used for receiving the glucose concentration, and the signal output end is configured to output a control signal matched with the glucose concentration.

The pump drive circuit module is respectively connected to the signal output end and an electrode of the second drug injection pump, and is used to output a driving current or a driving voltage matched with the control signal to the second drug injection pump.

The second drug injection pump is used for injecting insulin into the patient based on an electrochemical reaction under the drive of the driving current or the driving voltage.

As can be seen from the second technical solution, the closed-loop control insulin injection system includes a drug injection pump, a first sensor, a sensor circuit module, a pump drive circuit module, and a controller. The sensor circuit module is configured to monitor a glucose concentration of a patient through a current signal detected by the first sensor. The controller is connected to the sensor circuit module and used for receiving the glucose concentration and outputting a control signal matched with the glucose concentration to a pump drive circuit. The pump drive circuit module is connected to a pair of electrodes of the drug injection pump, and used to output a driving current or a driving voltage matching with the control signal to the drug injection pump. The drug injection pump is used to inject insulin into the patient under the drive of the driving current or driving voltage. As the system can automatically administer a drug to the patient based on the glucose concentration of the patient, the automation of drug administration process is achieved.

The closed-loop control insulin injection system is used to match the administration dosage with the glucose concentration of the patient, thus solving the problem that manual insulin injection cannot ensure that the glucose is within the normal range.

Third: the drug injection device based on the electrochemical reaction is a closed-loop control system based on a microneedle sensor. The closed-loop control system includes:
an electrochemical micropump, a second sensor, and a control module.

The electrochemical micropump includes a pump body. The pump body is provided with an accommodation region, the accommodation region is filled with media solution and is provided with an electrode layer connected to an inner wall of the pump body, and the pump body is provided with an expansion membrane covering the accommodation region.

The second sensor includes a substrate, a microneedle array, and an electrode overlying the substrate. The microneedle array is integrally molded with the substrate, and includes multiple hollow microneedles. Each hollow microneedle is internally provided with an injection channel.

The expansion membrane is connected to the substrate of the second sensor, and the tip of the hollow microneedle faces one side away from the expansion membrane.

An input end of the control module is connected to an output end of the second sensor, and an output end of the control module is connected to an input end of the electrochemical micropump. The control module is used to receive an electrical signal output by the second sensor and control the turn-on and turn-off of the electrochemical micropump according to the electrical signal.

As can be seen from the third technical solution, the closed-loop control system provided by the embodiment of the present disclosure is provided with an electrochemical micropump, a second sensor, and a control module. An expansion membrane of the electrochemical micropump is connected to a substrate of the second sensor, an input end of the control module is connected to an output end of the second sensor, and an output end of the control module is connected to an input end of the electrochemical micropump. The second sensor can be used to detect a glucose concentration in subcutaneous tissue fluid of a patient. Due to the fact that the glucose concentration of the tissue fluid has a strong correlation with the blood glucose concentration, a signal output by the second sensor can reflect the blood glucose concentration. Meanwhile, the second sensor can output a signal to the control module, and the control module can further control the turn-on and turn-off of the electrochemical micropump according to the electrical signal output by the second sensor, thus making the electrochemical micropump inject insulin according to the blood sugar concentration of the patient in real time and achieving automatic drug administration.

In the closed-loop control system provided by the embodiment of the present disclosure, the defect of large volume of the existing closed-loop control system is overcome by using the electrochemical micropump with small volume and the integrated arrangement of the electrochemical micropump and the sensor overcome. Moreover, in accordance with the closed-loop control system provided by the embodiment of the present disclosure, the used material and the processing method are improved, and the cost of the closed-loop control system is reduced.

In order to achieve the above objective, the embodiment of the present disclosure further provides the following solution:

A fabrication method for a drug injection pump is provided, which is used for fabricating the drug injection pump above. The fabrication method includes the steps of:
fabricating an electrode on a substrate;
bonding a driving cavity to the substrate to completely cover the electrode, and perfusing an electrolyte in the driving cavity; and
forming a drug storage component in the substrate, and enabling the drug storage component to completely encase the driving cavity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a schematic diagram of a drug injection pump based on an electrochemical reaction according to an embodiment II of the present disclosure;

FIG. 2 is a schematic diagram of another drug injection pump based on an electrochemical reaction according to an embodiment II of the present disclosure;

FIG. 3 is a schematic diagram of still another drug injection pump based on an electrochemical reaction according to an embodiment II of the present disclosure;

FIG. 4 is a schematic diagram of still another drug injection pump based on an electrochemical reaction according to an embodiment II of the present disclosure;

FIG. 5 is a schematic diagram of still another drug injection pump based on an electrochemical reaction according to an embodiment II of the present disclosure;

FIG. 6 is a schematic diagram of still another drug injection pump based on an electrochemical reaction according to an embodiment II of the present disclosure;

FIG. 7 is a schematic diagram of a curved substrate according to an embodiment II of the present disclosure;

FIG. 8 is a schematic diagram of a serrated substrate according to an embodiment II of the present disclosure;

FIG. 9 is a schematic diagram of another curved substrate according to an embodiment II of the present disclosure;

FIG. 10 is a schematic diagram of a hollow microneedle-shaped substrate according to an embodiment II of the present disclosure;

FIG. 11 is a schematic diagram of a wrinkled substrate according to an embodiment II of the present disclosure in a normal state;

FIG. 12 is a schematic diagram of a wrinkled substrate according to an embodiment II of the present disclosure in a stretching state;

FIG. 13 is a schematic diagram of still another drug injection pump based on an electrochemical reaction according to an embodiment II of the present disclosure;

FIG. 14 is a top view of a platinum interdigital electrode according to an embodiment II of the present disclosure;

FIG. 15 is a schematic diagram of a plate electrode according to an embodiment II of the present disclosure;

FIG. 16 is a schematic diagram of another plate electrode according to an embodiment II of the present disclosure;

FIG. 17 is a schematic diagram of a pillar electrode according to an embodiment II of the present disclosure;

FIG. 18 is a flow chart of a fabrication method for a drug injection pump in accordance with an embodiment II of the present disclosure;

FIG. 19 is a schematic diagram of a closed-loop control insulin injection system according to an embodiment IV of the present disclosure;

FIG. 20 is a schematic diagram of another closed-loop control insulin injection system according to an embodiment IV of the present disclosure;

FIG. 21 is a block diagram of a closed-loop control insulin injection system according to an embodiment IV of the present disclosure;

FIG. 22 is a schematic diagram of a wrinkled substrate according to an embodiment IV of the present disclosure in a natural state;

FIG. 23 is a schematic diagram of a sensor electrode according to an embodiment IV of the present disclosure;

FIG. 24 is a schematic diagram of another sensor electrode according to an embodiment IV of the present disclosure;

FIG. 25 is a structure schematic diagram of a closed-loop control system based on a microneedle sensor according to an embodiment V of the present disclosure;

FIG. 26 is a structure schematic diagram of another closed-loop control system based on a microneedle sensor according to an embodiment V of the present disclosure;

FIG. 27 is a schematic diagram of an electrochemical micropump with a serrated substrate according to an embodiment V of the present disclosure;

FIG. 28 is a structure schematic diagram of an electrochemical micropump with a curved substrate according to an embodiment V of the present disclosure;

FIG. 29 is a structure schematic diagram of a closed-loop control system including a first conversion subunit, a control subunit and a second conversion subunit according to an embodiment V of the present disclosure;

FIG. 30 is a structure schematic diagram of a sensor with a counter electrode as a power supply electrode in a closed-loop control system according to an embodiment V of the present disclosure;

FIG. 31 is a structure schematic diagram of a sensor with a reference electrode and a counter electrode as power supply electrodes in a closed-loop control system according to an embodiment V of the present disclosure;

FIG. 32 is a schematic diagram of a sensor with a conical hollow microneedle in a closed-loop control system according to an embodiment V of the present disclosure;

FIG. 33 is a schematic diagram of the microneedle array inserted into the dermis of the skin and interstitial fluid to perform glucose sensing and insulin releasing with a refillable electrochemical micropump;

FIG. 34 is a schematic diagram of the signal transduction (orange), conditioning, processing (blue) and transmission (green) paths for the closed-loop control of blood glucose level;

FIG. 35 is a schematic diagram of the multilayer structure of the biosensor and the biosensing principle;

FIG. 36 is a illustration of the fabrication process of the TPU microneedle array biosensor;

FIG. 37 shows a load-displacement curve on the TPU substrate by the in-situ nanomechanical test system;

FIG. 38 shows the sensitivity of the biosensor in the sensing H₂O₂ after the deposition of different cycles of PB (n=3);

FIG. 39 shows the Nyquist plots of the Au working electrode with different thicknesses of PB layers;

FIG. 40 shows the cyclic voltammograms of the biosensor with different thicknesses of PB layer in the 0.1 M KCl/HCl solution;

FIG. 41 shows the cyclic voltammograms curves of the biosensor for sensing 4 mM H₂O₂ in PBS at different scan rates;

FIG. 42 shows the sensitivities of the biosensor for detecting 4 mM H₂O₂ in PBS under different potentials;

FIG. 43 shows the current-verses-time response and calibration curve (n=3) upon the additions of H₂O₂ in PBS on the biosensor; where C1: 0.8 mM, C2: 2.2 mM, C3: 4.0 mM, C4: 5.0 mM, C5: 10 mM, C6: 12 mM, C7: 14 mM;

FIG. 44 shows the OCP curves of the Ag/AgCl electrode with different chlorination time in 3 M KCl solution;

FIG. 45 shows the current baseline response and calibration curve for detecting glucose in simulated interstitial fluid (n=3);

FIG. 46 show the influence of different volumes of the Insulin Aspart injection solution to the biosensor;

FIG. 47 shows the pH stability of the sensor over pH 6 to 8 (each glucose response was measured in PBS with different pH values, n=3);

FIG. 48 shows the temperature stability of the sensor at 20 to 40° C. (each glucose response was measured in PBS on the hot plate, n=3);

FIG. 49 shows the storage stability of the sensor over 10 days;

FIG. 50 shows the influence of bending on detecting glucose (bending angle: 45°, n=3);

FIG. 51 shows the stability of the sensor after different bending times (bending angle: 45°, n=3);

FIG. 52 shows the stability of the sensor after being bended at different angles (n=3);

FIG. 53 shows a schematic diagram of the working principle of the electrochemical micropump;

FIG. 54 shows a scheme of the fabrication and working process of the refillable electrochemical micropump;

FIG. 55 shows an EIS analysis of the Pt interdigital electrodes after applying different constant currents for 10 mins (the EIS measurement was conducted in deionized water);

FIG. 56 shows the flow rates of the micropump for releasing deionized water under different currents (n=3);

FIG. 57 shows the potential needed of the micropump at different currents from 0 to 2.0 mA (the insert image shows the potential-time curve under 0.4-2.0 mA, each current was measured for 60 s;

FIG. 58 shows the power needed of the micropump at different currents and the change of power/flow rate;

FIG. 59 shows the flow rates of the micropump for releasing deionized water and different concentrations (10 U/ml and 100 U/ml) of the fast-acting Insulin Aspart Injection solution at 1 mA in 40 mins;

FIG. 60 shows the flow rate change of the micropump for releasing deionized water with different distances between the adjacent two fingers of interdigital electrodes at the currents of 0.4-2.0 mA (n=3);

FIG. 61 shows the flow rate change of the micropump for releasing deionized water with different areas of interdigital electrodes at the currents of 0.4-2.0 mA (n=3);

FIG. 62 show the potential needed of the micropump with different areas of interdigital electrodes at the currents of 0.4-2.0 mA;

FIG. 63 shows an illustration of the system applied to the diabetic rat;

FIG. 64 shows the working model of the system applied to the rat (the final current in the first blue line was −7.43 μA, corresponding to a blood glucose level 16.3 mM; the final current in the second blue line was −5.29 μA, corresponding to a blood glucose level of 8.1 mM; insulin is 30 U/ml;

FIG. 65 shows the blood glucose levels monitored by a clinically approved blood glucose meter and the microneedle biosensor;

FIG. 66 shows a Clark rigor grid for this biosensor X-axis represents blood glucose values measured by the commercial blood glucose meter, Y-axis displays the glucose values measured by the biosensor (the data was from six different diabetic rats);

FIG. 67 shows the changing trend of the rat's blood glucose level (%) under different conditions (the error bar of blue, green purple lines was from three different rats, the error bar of the red line was from six different rats);

FIG. 68 show the change of the blood glucose level after stopping the closed-loop system with and without a glucose intake;

FIG. 69 shows a comparison of the closed-loop measurement of the blood glucose level under different conditions with a glucose intake;

FIG. 70 shows another form of the electrochemical micropump to form the closed-loop system;

FIG. 71 shows a fabrication process of the micropump;

FIG. 72 shows a schematic diagram of PCB for the biosensor and micropump;

FIG. 73 show a power delivery diagram of the system;

FIG. 74 shows cyclic voltammograms for electrodepositing and stabilizing Prussian blue layer on the Au electrode;

FIG. 75 shows magnified image of the Nyquist plot in low impedance;

FIG. 76 shows a Bode plot of impedance;

FIG. 77 shows a Bode plot of phase;

FIG. 78 shows the oxidation peak currents of cyclic voltammograms of the biosensor with different thicknesses of PB layer in the 0.1 M KCl/HCl solution;

FIG. 79 show the cyclic voltammograms of the biosensor before and after the deposition of Prussian blue, and Nafion membrane in PBS containing 4 mM H2O2 (scanning rate: 100 mV/s);

FIG. 80 shows a relationship between peak currents and the square root of scan rates in the cyclic voltammograms curves of the biosensor for sensing 4 mM H₂O₂ in PBS at different scan rates;

FIG. 81 shows a relationship between peak currents and the square root of scan rates in the cyclic voltammograms curves of the biosensor for sensing 4 mM H₂O₂ in PBS at different scan rates;

FIG. 82 show the cyclic voltammograms and calibration curve of the biosensor for sensing 4 mM glucose in PBS at different scan rates;

FIG. 83 shows a selective response of the sensor to glucose and other interfering substances (UA: uric acid, AA: ascorbic acid);

FIG. 84 shows the repeatability study was conducted by continuously measuring the glucose for 40 times;

FIG. 85 shows a Nyquist plot of the Pt interdigital electrode after the application of different constant currents for 10 mins in scan frequency from 1×10⁻² to 1×10⁵ Hz;

FIG. 86 show a Bode plot of the Pt interdigital electrode after the application of different constant currents for 10 mins in scan frequency from 1×10⁻² to 1×10⁵ Hz;

FIG. 87 shows flow rate of the refillable electrochemical micropump with shape 1 and size 1 of the chamber;

FIG. 88 shows flow rate of the refillable electrochemical micropump with shape 2 and size 2 of the chamber;

FIG. 89 shows flow rate of the refillable electrochemical micropump with shape 3 and size 3 of the chamber;

FIG. 90 shows flow rate of the refillable electrochemical micropump with shape 4 and size 4 of the chamber;

FIG. 91 shows flow rate of the refillable electrochemical micropump with shape 5 and size 5 of the chamber;

FIG. 92 shows flow rate of the refillable electrochemical micropump with shape 6 and size 6 of the chamber;

FIG. 93 shows the temperature stability of the micropump at 20 to 50° C. (n=3);

FIG. 94 shows the storage stability of the sensor over 14 days (n=3);

FIG. 95 shows the relative error of the biosensor at different blood glucose values (the data was from six diabetic rats) according to an embodiment;

FIG. 96 shows the relative error of the biosensor at different blood glucose values (the data was from six diabetic rats) according to another embodiment;

FIG. 97 shows the blood glucose levels versus time with the closed-loop button-like system in another two rats without a glucose intake;

FIG. 98 shows the blood glucose levels versus time with the closed-loop button-like system in another two rats with the injection of glucose intraperitoneally; and

FIG. 99 shows the blood glucose levels versus time with the closed-loop button-like system with a glucose intake in a terminal-stage diabetic rat (the model time was one month ago).

In the drawings: 1—driving component, 2—drug storage component, 3—liquid outlet hole, 4—catheter, 5—liquid injection hole, 6—movable plug body, 7—wire, 8—driving cavity, 9—microtube;
10—first sensor, 11—square tubular structure, 102—hexagonal tubular structure, 103—sensor electrode of first sensor;
11—electrochemical micropump, 111—pump body, 112—media solution, 113—micropump electrode, 114—expansion membrane, 115—micropump positive electrode, 116—micropump negative electrode, 13—drug injection pump;
12—second sensor, 121—microneedle array, 1211—hollow microneedle, 122—electrode of second sensor, 1221—working electrode, 1222—counter electrode, 1223—reference electrode, 123—substrate;
20—control apparatus, 30—controller, 301—signal input end, 302—signal output end, 40—sensor circuit module, 50—pump drive circuit module, 60—control module, 601—first conversion subunit, 602—second conversion subunit, 603—control subunit.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

An objective of the present disclosure is to provide a drug injection device based on an electrochemical reaction and a fabrication method for a drug injection pump, so as to solve the problem of automation in the drug administration process.

To make the objectives, features and advantages of the present disclosure more apparently and understandably, the following further describes the present disclosure in detail with reference to the accompanying drawings and the specific embodiments.

### Embodiment I

It is provided a drug injection device based on an electrochemical reaction according to an embodiment of the present disclosure. The drug injection device based on an electrochemical reaction is used to generate a driving force based on an electrochemical reaction, and then to automatically drive drug solution under the driving force to administer a drug to a patient.

### Embodiment II

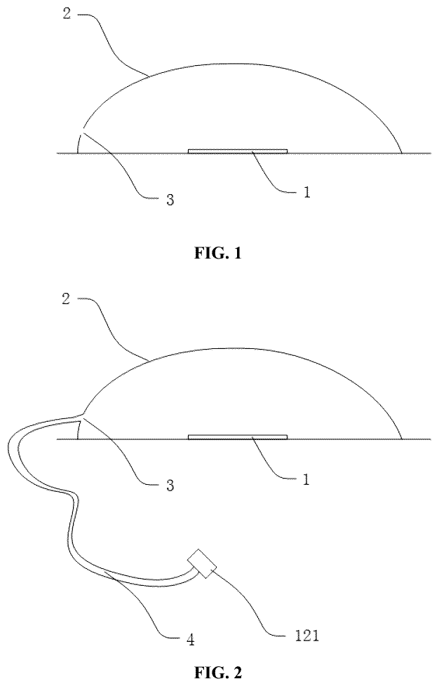

The drug injection device based on the electrochemical reaction provided by Embodiment I is a first drug injection pump based on electrochemical reaction. FIG. 1 is a schematic diagram of a drug injection pump based on an electrochemical reaction according to an embodiment of the present disclosure.

As shown in FIG. 1, the drug injection pump provided in this embodiment includes a driving component 1 and a drug storage component 2. The driving component is arranged inside the drug storage component.

The drug storage component is a component with a cavity, in which drug solution for injection is loaded, and the drug storage component is provided with at least one liquid outlet hole 3. The driving component is arranged inside the drug storage component, and the driving component is used to generate a drive force based on an electrochemical principle, i.e., based on a current input by an external control apparatus.

Under the action of the driving force generated by the driving component, the drug storage component discharges the drug solution through the liquid outlet hole by means of deformation or pressure change, and the liquid outlet hole is used to achieve the injection to a patient through a catheter 4 and a microneedle array 121, as shown in FIG. 2. When the drug injection pump of the present disclosure is arranged inside a human body, the liquid outlet hole can administer a drug to the patient without additional elements.

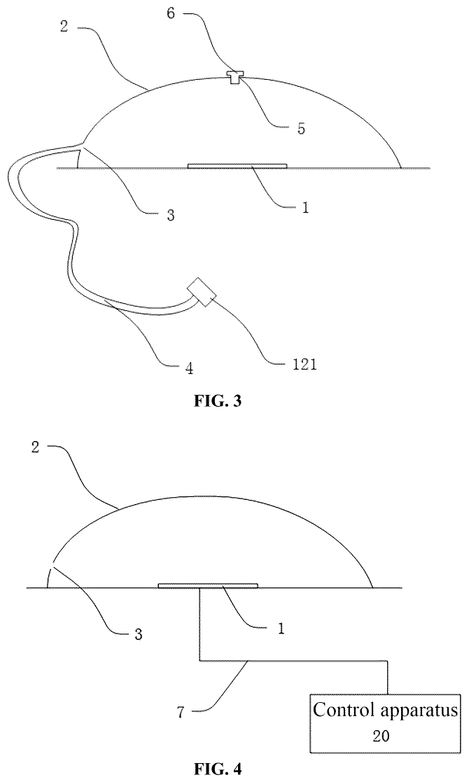

In a specific embodiment of the present disclosure, the corresponding part of the drug storage cavity is provided with a liquid injection hole 5 in addition to the liquid outlet hole. As shown in FIG. 3, the drug solution can be filled into the drug storage cavity through the liquid injection hole, and the sealing can be achieved through a movable plug body 6 after the filling is completed.

A diameter of the liquid injection hole is from 1 mm to 5 mm, and a diameter of the liquid outlet hole is from 1 mm to 5 mm. In this embodiment, the diameter of the liquid injection hole is preferably 3 mm, and the diameter of the liquid outlet hole is also preferably 3 mm. The size of the liquid injection hole and the liquid outlet hole may also be micron-sized.

The driving component is used to generate the driving force based on an electrochemical reaction principle. The electrochemical element is connected to the outside of the drug storage component via a wire 7, and is used to receive a current output from the control apparatus 20, as shown in FIG. 4, and to generate a gas based on the current, and thus the driving force on the drug solution in the drug storage component is generated through volume expansion. A driving voltage ranges from 0.1 volts to 20 volts, which is the normal withstand voltage of the human body and will not cause harm to human body. In addition, the driving voltage may also be a constant driving current from 0.1 mA to 10 mA.

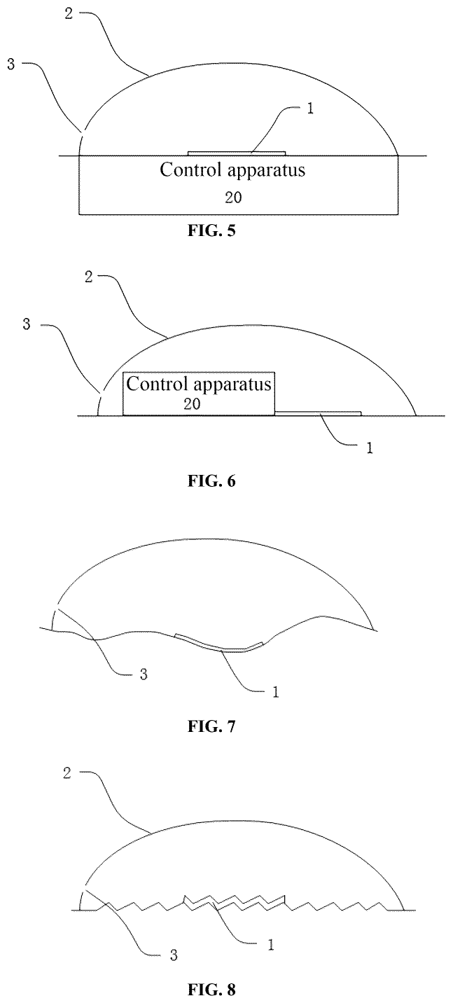

In addition, as described above, the drug injection pump of the present disclosure may be arranged inside the human body. At the moment, the control apparatus with a battery can be integrated with the pump, as shown in FIG. 5 and FIG. 6, so as to achieve long-term autonomous operation in the human body.

The driving component includes an electrochemical element. The electrochemical element includes at least one pair of electrodes connected to the control apparatus via wires. The electrode is a metal electrode or a composite conductive material electrode. The metal electrode may be made of platinum, gold, silver, copper, etc., and a carbon electrode may also be employed. Based on the current, the electrode enables the water or other components in the drug solution to undergo redox reaction or electrolytic reaction, so as to generate a corresponding gas.

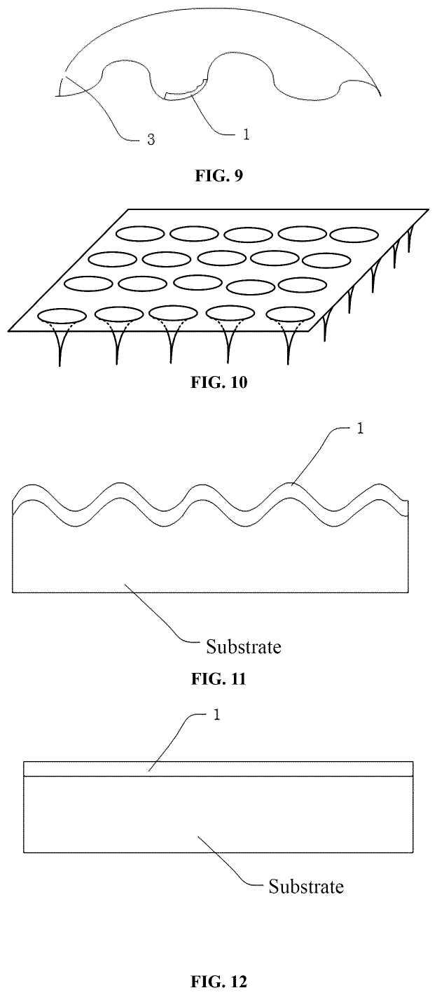

The electrode is arranged on a substrate. The substrate can be regarded as a part of the electrode, and is used for bearing an electrode material. The substrate may have a planar surface, a curved surface, a serrated shape, or a micro-needled surface, as shown in FIG. 7, FIG. 8, FIG. 9 and FIG. 10. The substrate may also have a wrinkled surface on which to fabricate the electrodes, as shown in FIG. 11. After the substrate is stretched, the electrode is deformed accordingly, as shown in FIG. 12. When choosing the material of the substrate, a flexible material may be used as the substrate, a hard material such as glass may also be used as the substrate, and an elastic substrate that can be stretched may also be employed.

As can be seen from the above technical solution, the drug injection pump based on the electrochemical reaction provided by the embodiment includes a driving component and a drug storage component. The driving component is located inside the drug storage component and is used to generate a driving force inside the drug storage component by utilizing a current based on an electrochemical principle. The drug storage component is internally loaded with drug solution, and is used to push out the drug solution and inject the drug solution into a patient under the push of the driving component. As the electrochemical reaction may be generated based on a preset regular electrical signal, the patient can be injected on time by controlling an input current or voltage, thus ensuring the stability, intelligence and automation of drug administration.

The drug in the drug injection pump in the present disclosure may be insulin injection, the concentration of which may be controlled at 1 U/ml to 500 U/ml, such that the drug injection pump becomes an insulin pump capable of carrying out automatic insulin supplementation for diabetic patients.

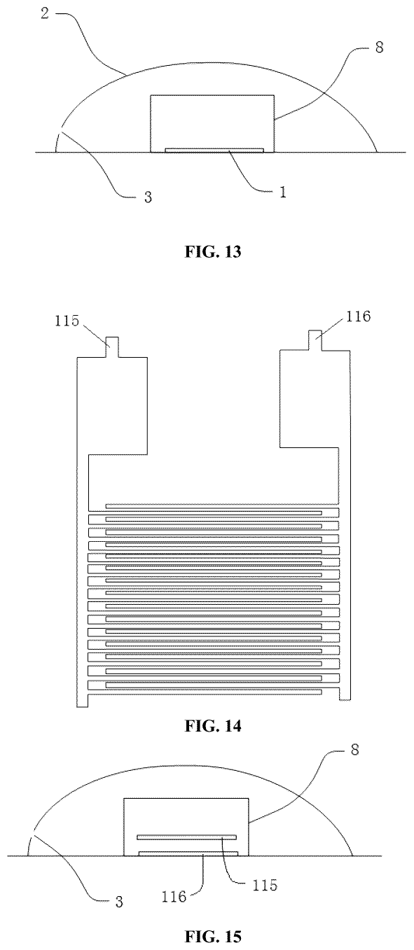

In another embodiment of the present disclosure, the driving component further includes a driving cavity 8, as shown in FIG. 13. The driving cavity is located inside the drug storage component and covers the electrochemical element, and an electrolyte is loaded inside the driving cavity, and the electrolyte undergoes redox reaction under the action of the electrochemical element, and the generated gas may expand the driving cavity, thereby generating a driving force for the drug solution in the drug storage component. The electrolyte may be pure water, salt solution, etc.

The driving cavity may be made of polytetrafluoroethylene, polydimethylsiloxane PDMS, polyacrylate, silica gel, rubber, latex, polyurethane, parylene, or polyimide.

The electrochemical element is preferably a platinum interdigital electrode. The platinum interdigital electrode includes interdigitated platinum electrode sheets. As shown in FIG. 14, a width of the platinum electrode sheet is 100 μm, and the distance between the platinum electrode sheets is also 100 μm. The platinum interdigital electrode is connected to the outside of the drug storage component via a wire, and is used for receiving a current via the wire, please referring to FIG. 14 for a micropump positive electrode 115 and a micropump negative electrode 116 of the platinum interdigital electrode.

The overall area of the electrode in the electrochemical element is from 1 square millimeter to 1 square centimeter, and the thickness of the electrode is from 50 nm to 500 μm in general. The electrode may be fabricated by sputtering or evaporation by micro-nanomachining, and may be made of platinum or gold and other chemically stable materials, or may be fabricated by screen printing.

The driving cavity is produced by assembling a bottom plate and a cover plate, and edges of the bottom plate and the cover plate are bonded together by bonding, thereby forming a cavity therein for accommodating an electrolyte and the electrochemical element. The cover plate is preferably 8 mm in diameter and 7 mm in height.

The drug storage component in this embodiment may be generated by a 3D printing process, an injection molding process or other processes, and is preferably made of Teflon material, and a thickness of a film is preferably 30 μm. The drug storage component has a diameter of 20 mm and a height of 10 mm.

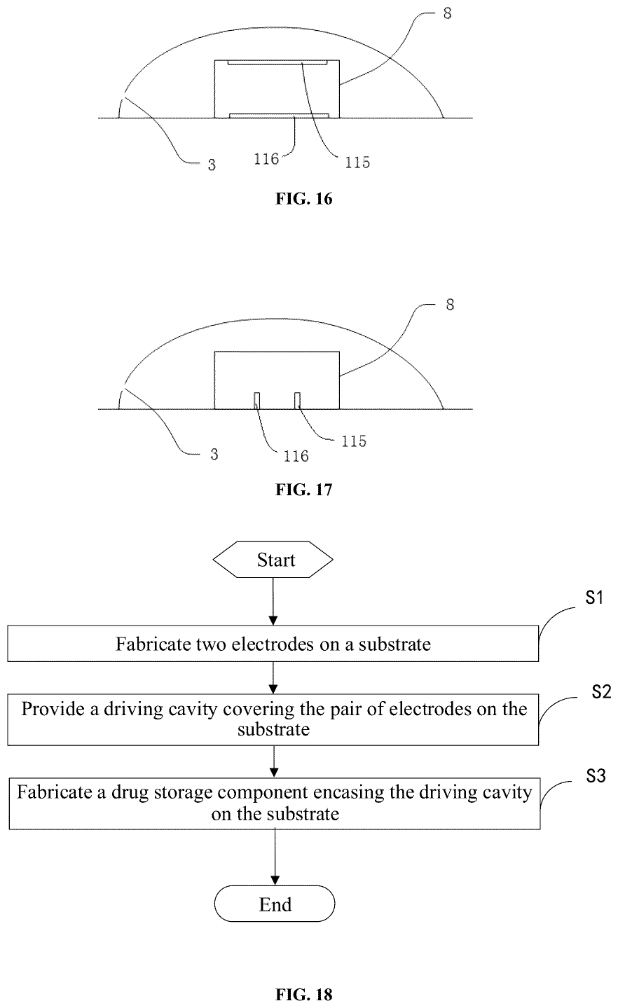

In addition, in addition to the platinum interdigital electrode, the electrode in this embodiment may also be in the form of two plate electrodes opposite from top to bottom, where one plate electrode is located on a substrate and the other plate electrode is located above the substrate, as shown in FIG. 15, and the other plate electrode may also be located on an inner wall of the driving cavity, as shown in FIG. 16. In FIG. 15 and FIG. 16, one of the two plate electrodes serves as a micropump positive electrode 115, and the other serves as a micropump negative electrode 116.

In addition, the electrode may also be two or two groups of electrodes located on the substrate and standing upright, as shown in FIG. 17. In FIG. 17, one of the two electrodes serves as a micropump positive electrode 115, and the other serves as a micropump negative electrode 116.

### Embodiment III

FIG. 18 is a flow chart of a fabrication method for a drug injection pump in accordance with an embodiment of the present disclosure.

Referring to FIG. 18, a fabrication method for a drug injection pump provided in this embodiment is also used for fabricating the drug injection pump provided in the previous embodiment. The method specifically includes the following steps:

S1. Two electrodes are fabricated on a substrate.

Specifically, the electrodes are fabricated on the substrate by a magnetron sputtering process, a screen-printing process, a metal evaporation process or other processes. The substrate then becomes the fabrication basis of the whole drug injection pump. The substrate may be made of glass, plastic, polyethylene glycol terephthalate PET, polyimide, polyurethane, polycarbonate, polyester, thermoplastic polyurethane TPU elastomer, polyvinyl chloride PVC, chitosan, polylactic acid, silica gel, rubber, latex, thermoplastic elastomer TPE, perfluoroethylene propylene copolymer FEP, and polytetrafluoroethylene PTFE.

The electrode is a platinum interdigital electrode, a carbon interdigital electrode, a gold interdigital electrode, or a composite conductive material interdigital electrode, i.e., an interdigital electrode made of a platinum material, a carbon material, a gold material or a composite conductive material, respectively. In this embodiment, the interdigital electrode is described by taking the platinum interdigital electrode as an example, the platinum interdigital electrode includes a titanium layer attached to a substrate and a platinum layer attached to the titanium layer.

The size of the substrate is 2 cm×2 cm. During fabrication, a titanium (Ti) film and a platinum (Pt) film are deposited on a glass substrate in turn, and then the fabrication of the platinum interdigital electrode is achieved by photolithography, i.e., the redundant film is removed in a manner of coating photoresist, photolithography, etching, thus forming the platinum interdigital electrode. On the basis of forming the corresponding electrode, the polarity of the electrode is determined by enabling the electrode to enter deionized water with acetone and isopropanol in solution.

S2. A driving cavity covering the electrodes is provided on the substrate.

Specifically, the driving cavity is adhered to the substrate by a sealant to cover the electrode.

S3. A drug storage component encasing the driving cavity is fabricated on the substrate.

Finally, the drug storage component made of a Teflon material is produced on the substrate by a 3D printing process, an injection molding process or other processes, thus completely encasing the driving cavity.

The fabrication of the drug injection pump can be achieved through the above arrangement.

### Embodiment IV

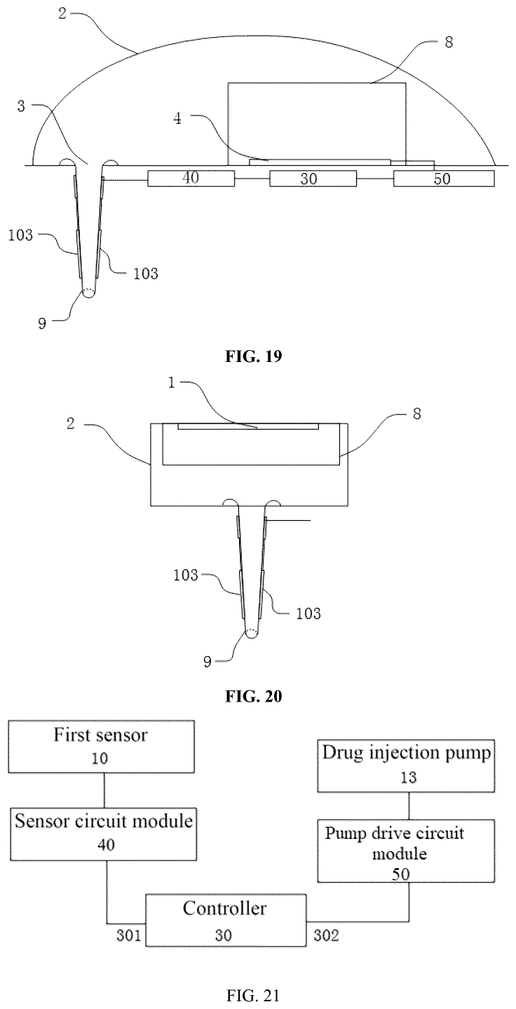

The drug injection device based on the electrochemical reaction of Embodiment I is a closed-loop control insulin injection system based on a microtube sensor. FIG. 19 is a schematic diagram of a closed-loop control insulin injection system according to an embodiment of the present disclosure.

The insulin injection system provided in this embodiment is used for regular and quantitative injection of insulin for a patient needing insulin injection, so as to keep the glucose of the patient within a normal range. The system includes a drug injection pump 13, a pump drive circuit module 50, a controller 30, a sensor circuit module 40, and a first sensor 10.

Referring to FIG. 19, the drug injection pump of the specific structure of the closed-loop control insulin injection system is provided with at least one microtube 9, one end of the microtube is connected to the interior of a drug storage component, and the other end of the microtube is used for entering the subcutaneous of a patient, so as to facilitate insulin in the drug storage component to enter the body of the patient under the drive of the driving component. A sensor electrode 103 of a first sensor is provided on an outer wall of the microtube and connected to the sensor circuit module 40. The pump drive circuit module 50, the sensor circuit module 40 and the controller 30 are provided at a lower part of the drug injection pump.

The microtube 9 and the first sensor 10 constitute a microtube sensor.

The closed-loop control insulin injection system may also be in the shape shown in FIG. 20, and its final shape is a button, which is convenient for a patient to wear.

As shown in FIG. 21, the controller is provided with a signal input end 301 and a signal output end 302. The signal input end is connected to the sensor circuit module, the signal output end is connected to the pump drive circuit module, and the pump drive circuit module is also connected to the drug injection pump.

The first sensor is arranged on the skin of the patient and is used for detecting a glucose concentration of the patient through a sensor electrode thereof. The sensor electrode can generate a current signal by detecting the subcutaneous tissue fluid and output the current signal to the sensor circuit module. The sensor circuit module is connected to the first sensor, and is used for generating a glucose concentration based on the current signal and outputting the glucose concentration to the signal input end of the controller through an output end of the sensor circuit module. After receiving the glucose concentration, the controller processes the glucose concentration, that is, determines parameters such as injection frequency and injection time according to the glucose concentration, and outputs a control signal to the pump drive circuit module at a predetermined time based on the above parameters. After receiving the control signal, the pump drive circuit module outputs driving power to the drug injection pump.

In general, an output signal of the first sensor is a micro-voltage signal from −0.1 volts to 0.6 volts, and its voltage range and voltage fluctuation have no adverse effects on human body.

The drug injection pump includes an electrode. The electrode is connected to the pump drive circuit module and is used for generating a driving force based on an electrochemical reaction under the drive of a driving current or a driving voltage, and the insulin is injected into the body of the patient by the driving force. The driving voltage ranges from 0.1 volts to 20 volts, which is the normal withstand voltage of the human body and will not cause harm to human body.

As can be seen from above technical solution, the embodiment provides a closed-loop control insulin injection system. The system includes a drug injection pump, a first sensor, a sensor circuit module, a pump drive circuit module, and a controller. The sensor circuit module is configured to monitor a glucose concentration of a patient through a current signal detected by the first sensor. The controller is connected to the sensor circuit module and used for receiving the glucose concentration and outputting a control signal matched with the glucose concentration to a pump drive circuit module. The pump drive circuit module is respectively connected to an electrode of the drug injection pump and used to output a driving current or a driving voltage matched with the control signal to the drug injection pump. The drug injection pump is used to inject insulin into the patient under the drive of the driving current or driving voltage. The system can be used to automatically administer a drug to the patient based on the glucose concentration of the patient, and the dosage is matched with the glucose concentration of the patient, thus solving the problem that manual insulin injection cannot ensure that the glucose is within the normal range.

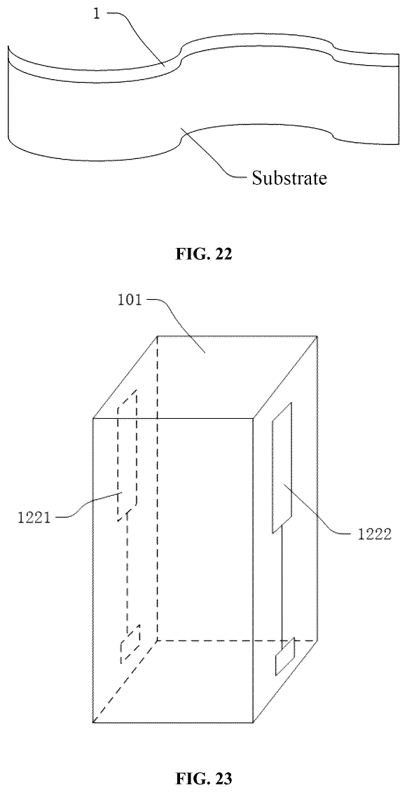

In a specific embodiment of the present disclosure, the drug injection pump has the same structure as the drug injection pump of Embodiment II, and its working principle and beneficial effect are similar, and will not be described in detail herein. The specific content can be referred to the introduction of the drug injection pump of Embodiment II. In this embodiment, FIG. 22 also shows a substrate bent and deformed in a natural state.

In this embodiment of the present disclosure, the first sensor includes a tubular structure and a sensor electrode. The sensor electrode is connected to the sensor circuit module, and is used to output a current signal to the module under the drive of a driving voltage output by the sensor circuit module. The cross section of the tubular structure may be square, hexagon, round or other shapes.

The tubular structure has a length from 1 mm to 20 mm, a diameter of an outer wall from 50 μm to 1,000 μm, and a thickness of a side wall from 10 μm to 200 μm. The sensor electrode has a width from 50 μm to 1,000 μm, a length from 1 mm to 15 mm and a thickness from 50 nm to 100 μm.

When the square tubular structure 101 is employed, the sensor electrodes 52 are disposed opposite to each other on the opposite side walls, as shown in FIG. 23. At this time, one of the two electrodes is a working electrode and the other is a reference electrode/counter electrode, which has the functions of both reference electrode and counter electrode. The working electrode may be a metal electrode, such as a gold electrode, or a platinum electrode, or may be a carbon electrode. The reference electrode/counter electrode may be a silver/silver chloride electrode.

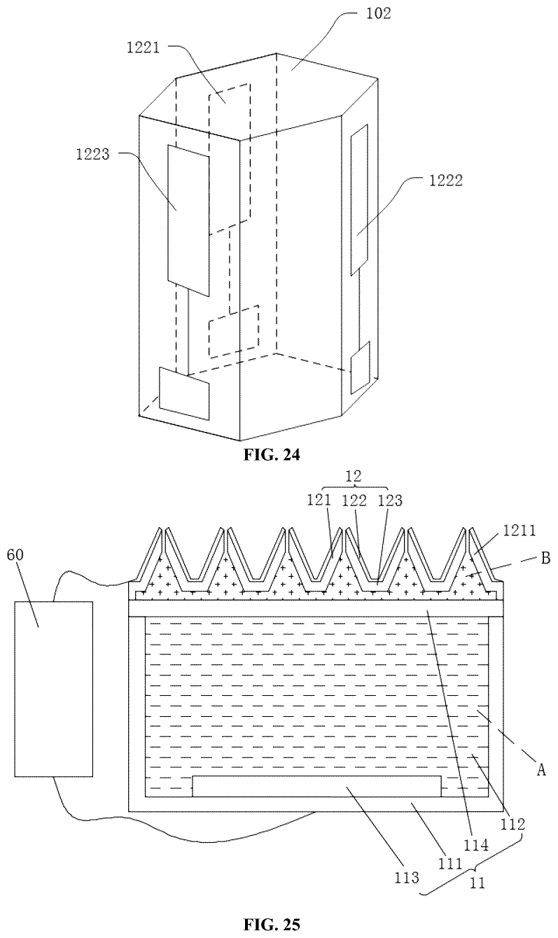

As shown in FIG. 24, when the tubular structure 102 having a hexagonal cross section is employed, the sensor electrodes are provided on adjacent side walls, which are a working electrode 1221, a reference electrode 1223, and a counter electrode 1222, respectively. The working electrode or the counter electrode may be a metal electrode, such as a gold electrode or a platinum electrode, or may be a carbon electrode. The reference electrode may be a silver/silver chloride electrode. Glucose oxidase is immobilized on the working electrode, which catalyzes glucose to produce hydrogen peroxide, which causes the change of current on the electrode. The working electrode may be made of gold, platinum, carbon and other materials plated with Prussian blue, or gold, platinum and carbon material combined with a layer of Prussian blue, and then the glucose oxidase is immobilized on the electrode and then is covered with a layer of biocompatible material. The reference electrode material may be silver/silver chloride, and the counter electrode may be made of gold, platinum or carbon.

The sensor electrode may be fabricated by a micromachining method or by screen printing.

The tubular structure is made of polyethylene glycol terephthalate, polyvinyl chloride, glass fiber, polyurethane, silk fibroin, chitosan, polylactic acid, polyimide, polyimide thermoplastic polyurethane elastomer, silica gel, rubber, latex, thermoplastic elastomer, perfluoroethylene propylene copolymer, or polytetrafluoroethylene.

The tubular structure may be 1 mm to 20 mm in length and 50 μm to 2 mm in diameter, such that the tubular structure can penetrate into the dermis or fat deposit of skin. The effect may be more remarkable by injecting insulin into the fat deposit.

The fabrication method for the drug injection pump provided by this embodiment is the same as that for the drug injection pump provided by Embodiment III, and will not be described in detail here. The specific content may be referred to the introduction of the fabrication method for the drug injection pump of Embodiment III.

### Embodiment V

The drug injection device based on an electrochemical reaction of Embodiment I is a closed-loop control system based on a microneedle sensor. Referring to FIG. 25, a closed-loop control system disclosed according to an embodiment of the present disclosure includes an electrochemical micropump 11, a second sensor 12, and a control module 60.

Specifically, as shown in FIG. 25, the electrochemical micropump 11 includes a pump body 111 having an accommodation region A in which a media solution 112 and a micropump electrode 113 (an electrode layer) are provided. The micropump electrode 113 is located on an inner wall of the pump body 111, and an expansion membrane 114 covering the accommodation region A is provided on the pump body 111. In an alternative embodiment, the pump body 111 may be cylindrical or hemispherical as a whole, the micropump electrode 113 may be an interdigital electrode made of a platinum material, the media solution 112 may be deionized water or salt solution, and the expansion membrane 114 may be a polytetrafluoroethylene membrane.

The interdigital electrode includes interdigitated platinum electrode sheets, the width of the platinum electrode sheet may be from 1 μm to 500 μm. The interdigital electrode is connected to the outside of the pump body 111 via a wire, and is used for receiving a current via the wire. In addition, the micropump electrode 113 may be made of gold, silver, aluminum, carbon, or the like. The area of the micropump electrode 113 may be from 1 mm² to 1 cm², the thickness of the micropump electrode 113 may be from 50 nm to 100 μm. When the micropump electrode 113 is an electrode of another shape such as a flat electrode, the width of the flat electrode may be in the range of millimeter to centimeter. The micropump electrode 113 may be formed on the substrate by a sputtering or evaporation process of micro-nano machining or may be formed on the substrate by screen printing.

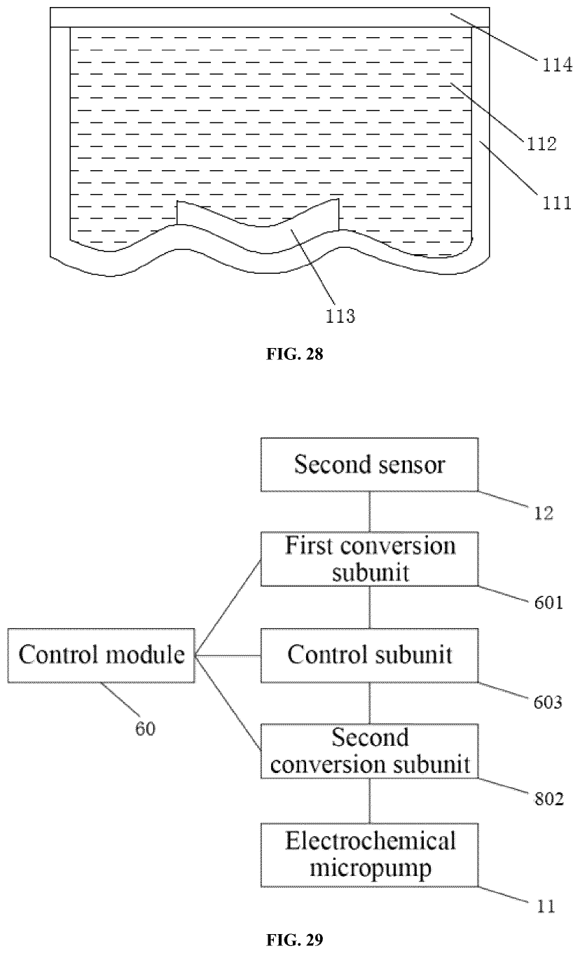

The inner wall of the pump body 111 where the micropump electrode 113 is located serves as the substrate of the micropump electrode 113. As shown in FIG. 25, FIG. 27 and FIG. 28, the shape of the substrate may be planar, serrated, or curved. The substrate may be made of a flexible material or a hard material such as glass, which is the same as the material of the substrate of Embodiment 3.

The expansion membrane 114 may also be made of polydimethylsiloxane (PDMS), polyacrylate, silica gel (e.g., Ecoflex, Dragon Skin), rubber (e.g., NBR, IIR), latex, polyurethane, parylene, polyimide, and other materials.

Referring to FIG. 25, the second sensor 12 includes a substrate 123, a microneedle array 121, and an electrode (electrode 122 of the second sensor) overlying the substrate 124. The microneedle array 121 is integrally molded with the substrate 123. The microneedle array 121 includes multiple hollow microneedles 1211. Each hollow microneedle 1211 is internally provided with an injection channel B, and the tip of the hollow microneedle 1211 is provided with an injection hole. After the second sensor 12 is connected to the expansion membrane 114, insulin solution can be injected into the injection channel B inside the hollow microneedle 1211. It should be noted that the aperture of the injection hole at the tip of the hollow microneedle 1211 is small, so that the insulin solution inside the hollow microneedle 1211 cannot flow out through the injection hole under the action of capillary force. The second sensor 12 is a microneedle sensor.

Specifically, a height of the hollow microneedle 1211 in the microneedle array 121 may be from 500 μm to 2,000 μm, a diameter of the hollow microneedle 1211 in the substrate 123 may be from 100 μm to 500 μm, and a thickness of a sidewall of the hollow microneedle 1211 may be from 30 μm to 300 μm. A thickness of the electrode 23 may be from 50 nm to 20 μm.

After the electrochemical micropump 11 is electrified, the micropump electrode 113 electrolyzes water to generate hydrogen bubbles and oxygen bubbles. These bubbles move towards the position where the expansion membrane 114 is located, and under the action of these bubbles, the expansion membrane 114 deforms and expands to squeeze the insulin solution inside the hollow microneedle 1211, such that the insulin solution flows out through the injection hole, and then the insulin solution can be injected into the body of the patient when the second sensor 12 acts on the patient. When the electrochemical micropump 11 is not electrified, hydrogen and oxygen can be recombined to form water through the catalysis of the micropump electrode 113. At the moment, the expansion membrane 114 may shrink, making the insulin solution no longer flow out of the injection hole at the tip of the hollow microneedle 1211.

An input end of the control module 60 is connected to an output end of the second sensor 12, and an output end of the control module 60 is connected to an input end of the electrochemical micropump 11. Therefore, the control module 60 may receive an electrical signal output from the second sensor 12. Since the hollow microneedle 1211 on the second sensor 12 enters the body of the patient and is in contact with the subcutaneous tissue fluid of the patient, the glucose concentration of the subcutaneous tissue fluid of the patient can be detected. Meanwhile, the glucose concentration of the tissue fluid has a strong correlation with the blood glucose concentration, so the electrical signal output from the second sensor 12 can reflect the blood glucose concentration. Exemplarily, the second sensor 12 may detect a current at a constant voltage, and the magnitude of the current signal is proportional to the magnitude of the glucose concentration.

Then the control module 60 may control the turn-on or turn-off of the electrochemical micropump 11 according to the electric signal, i.e., the electrochemical micropump 11 is electrified or not. Exemplarily, a preset value may be set within the control module 60, the electrochemical micropump 11 is electrified in a case that a value of the electric signal is greater than or equal to the preset value, and the electrochemical micropump 11 is not electrified in a case that a value of the electric signal is less than the preset value.

In this way, the electrochemical micropump 11 may be controlled according to a real-time blood glucose concentration of the patient.

Further, the substrate 123 and the microneedle array 121 of the second sensor 12 may be fabricated using a mold with the shape of microneedle array. During specific fabrication, the substrate 123 can be formed by casting a liquid polymer material on the mold with the shape of the microneedle array 121 and demolding after drying. The liquid polymer material may be biodegradable materials, such as chitosan, polylactic acid and silk fibroin, or biocompatible materials, such as thermoplastic polyurethane. When the liquid polymer material is made of the biodegradable material, the microneedle sensor may have degradable ability and can be decomposed naturally after use. The use of biocompatible material makes the biocompatibility of the microneedle sensor stronger, and can avoid damage to human body when using.

In an alternative embodiment, the substrate and the microneedle array 121 of the second sensor 12 may also be fabricated by 3D printing. Specifically, the second sensor 12 may be made of epoxy resin, ceramic, metal, a biocompatible material, a biodegradable material, etc.

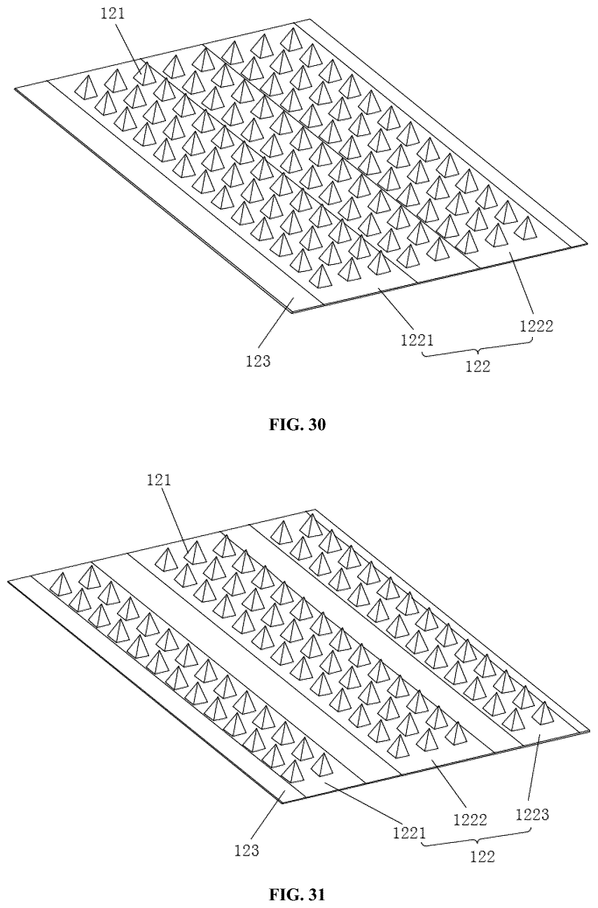

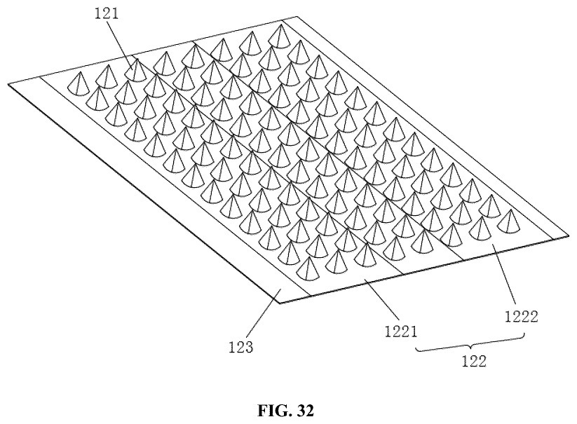

Further, with reference to FIG. 30 and FIG. 32, the shape of the hollow microneedle 1211 in the microneedle array 121 may be a pyramid or a cone, which is not specifically limited in the embodiment of the present disclosure. The injection hole at the tip of the hollow microneedle 1211 may be formed by penetrating a metal needle array. Specifically, a stainless-steel needle array can be used to penetrate the tip of each hollow microneedle 1211 in the microneedle array 121, thus making the tip of each hollow microneedle 1211 obtain an obvious hole for use as an injection hole.

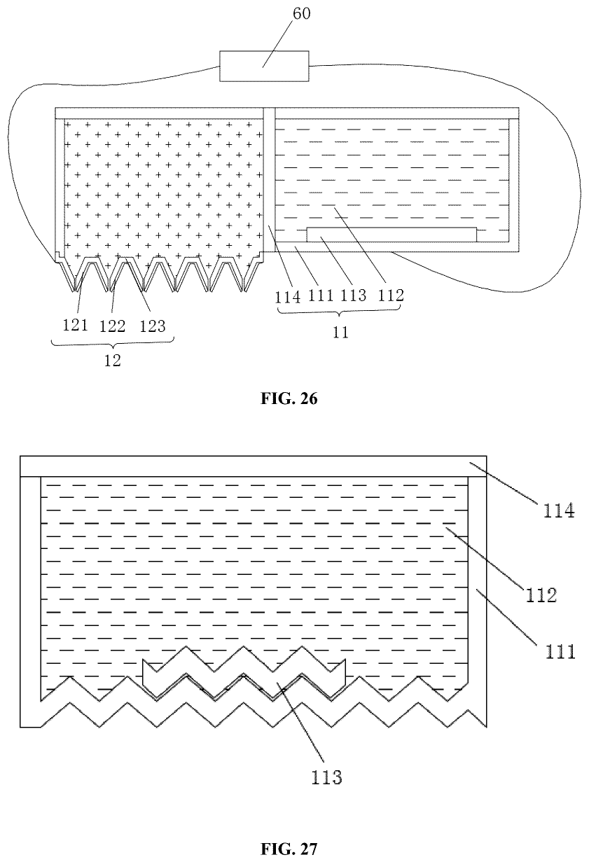

In an alternative embodiment, referring to FIG. 26, it is also provided another closed-loop control system according to an embodiment of the present disclosure. In the closed-loop control system, the second sensor 12 is located on the left side (or the right side) of the expansion membrane 114. In this embodiment, the space between the second sensor 12 and the expansion membrane 114 may be used to store more insulin solution, thereby further facilitating the use of the closed-loop control system.

In an alternative embodiment, referring to FIG. 29, the control module 60 includes a first conversion subunit 601, a control subunit 603, and a second conversion subunit 602.

Specifically, an input end of the first conversion subunit 601 is connected to an output end of the second sensor 12, and an output end of the first conversion subunit 601 is connected to an input end of the control subunit 32. The first conversion subunit 601 is used for receiving and converting an electrical signal output by the second sensor 12. Exemplarily, the second sensor 12 outputs a corresponding current signal after detecting the glucose concentration in the body of a patient, and the first conversion subunit 601 can detect the current signal and convert the current signal and transmit the current signal to the control subunit 603. Meanwhile, the first conversion subunit 601 may also supply a constant voltage to the second sensor 12, where the constant voltage may be different voltages such as 0.1 V, −0.1 V, or 0.6 V.

An input end of the second conversion subunit 602 is connected to an output end of the control subunit 603, and an output end of the second conversion subunit 602 is connected to an input end of the electrochemical micropump 11. After receiving the electrical signal converted by the first conversion subunit 601, the control subunit 603 sends a command to the second conversion subunit 602 according to the electrical signal. The second conversion subunit 602 can receive the command output by the control subunit 603, convert the command into a corresponding command signal, and then transmit the command signal to the electrochemical micropump 11, so as to control the turn-on and turn-off of the electrochemical micropump 11.

Exemplarily, the electric signal received by the control subunit 603 is greater than an electric signal with the preset value, so the control subunit 603 may output an electrifying command to the second conversion subunit 602, the second conversion subunit 602 receives the electrifying command and converts the command into an electrifying command signal, and then the electrochemical micropump 11 is electrified after receiving the electrifying command signal. Meanwhile, the second conversion subunit 602 may provide a constant voltage or a constant current to drive the electrochemical micropump 11 and further control the injection amount of insulin by controlling the magnitude and duration of the voltage, the voltage magnitude may be 0.1 V to 20 V, and the current magnitude may be 0.1 mA to 10 mA.

In this way, after the second sensor 12 detects the glucose concentration in the body of the patient and generates an electrical signal, the first conversion subunit 601 may receive and convert the electrical signal, and then send the converted electrical signal to the control subunit 603. After receiving the electrical signal converted by the first conversion subunit 601, the control subunit 603 may generate different commands according to the different electrical signals, meanwhile, the control subunit 603 sends the generated command to the second conversion subunit 602. The second conversion subunit 602 converts the received command into a corresponding command signal, and controls the electrochemical micropump 11 on or off according to the command signal, thus achieving the control of the electrochemical micropump 11 according to the real-time blood glucose concentration of the patient.

In an alternative embodiment, the first conversion subunit 601 is a first signal converter. The control subunit 603 is a microcontroller. The second conversion subunit 602 is a second signal converter.

Specifically, devices in the related art can be used as a first signal converter and a second signal converter by those skilled in the art, as long as the effect of controlling the turn-on or turn-off of the electrochemical micropump 11 can be achieved by the control module 60. Therefore, it is not specifically limited in this embodiment, and the specific contents of the related art are not repeated.

In an alternative embodiment, the closed-loop control system further includes a cloud server to which the control subunit 603 is electrically connected.

The cloud server is used to receive and store information sent by the control subunit 603. The information sent by the control subunit 603 may include the glucose concentration in the body of the patient.

In an alternative embodiment, the closed-loop control system further includes a display module. The display module is electrically connected to the control subunit 603, while the display module may also be connected to the cloud server.

The display module is used for receiving and displaying the information sent by the control subunit 603. During specific application, the display module may be a computer, a display, a tablet computer, etc.

In an alternative embodiment, the electrode 122 of the second sensor may include a working electrode 1221 and a power supply electrode.

Specifically, during fabrication, the working electrode 1221 and the power supply electrode may be fabricated on a protruding side of the microneedle array 121 on the substrate 123, or the working electrode 1221 and the power supply electrode may also be fabricated on a concave side of the microneedle array 121 on the substrate 123. When the working electrode 1221 and the power supply electrode are fabricated on the protruding side of the microneedle array 121, it is not necessary to penetrate the microneedle array 121 at the tip of the hollow microneedle 1211, because at this time, it is only necessary to make the microneedle contact with the detected solution, and there is no need for the detected solution to flow into the microneedle array 121.

Meanwhile, the working electrode 1221 includes an electrode layer, a Prussian blue layer, a reagent enzyme layer and a biocompatible polymer layer laminated on the substrate 123, in which the electrode layer may be made of gold, platinum or carbon, while the power supply electrode generally includes an electrode layer.

The reagent enzyme layer is covered with a liquid biocompatible polymer, and then the liquid biocompatible polymer is dried and heated to form a biocompatible polymer layer. The biocompatible polymer layer may be made of perfluorosulfonic acid, and the biocompatible polymer layer can prevent the Prussian blue layer from causing damage to the human body.

In this way, when the working electrode 1221 comes into contact with the detected solution, the reagent enzyme may react with the corresponding analyte in the detected solution, and a product is produced by the reagent enzyme reaction, which may undergo oxidation or reduction reaction on the working electrode 1221 cause electrical signal change.

During specific application, as shown in FIG. 30, the power supply electrode may only include a counter electrode 1222, at the moment, the counter electrode 1222 can play a role of connecting circuits and stabilizing a voltage simultaneously. The counter electrode 1222 may be made of silver/silver chloride. As shown in FIG. 31, the power supply electrode may include a reference electrode 1223 and a counter electrode 1222, where the reference electrode 1223 plays a role of stabilizing a voltage, and the counter electrode 1222 plays a role of connecting a circuit a voltage stabilizer and the counter electrode 1222 functions as a communication circuit. The counter electrode 1222 can be made of gold, platinum or carbon; The material of the reference electrode 1223 may be silver/silver chloride.

Further, the drug injection device based on an electrochemical reaction of Embodiment I is a closed-loop system for insulin injection, including the closed-loop control system as provided in the embodiment of the present disclosure.

Specifically, the closed loop system for insulin injection further includes an insulin delivery device for delivering insulin. An output end of the insulin delivery device is located in a region between the second sensor 12 and the expansion membrane 114, such that the electrochemical micropump 11 can control the precise injection of insulin according to the blood glucose concentration of a patient detected by the second sensor 12.

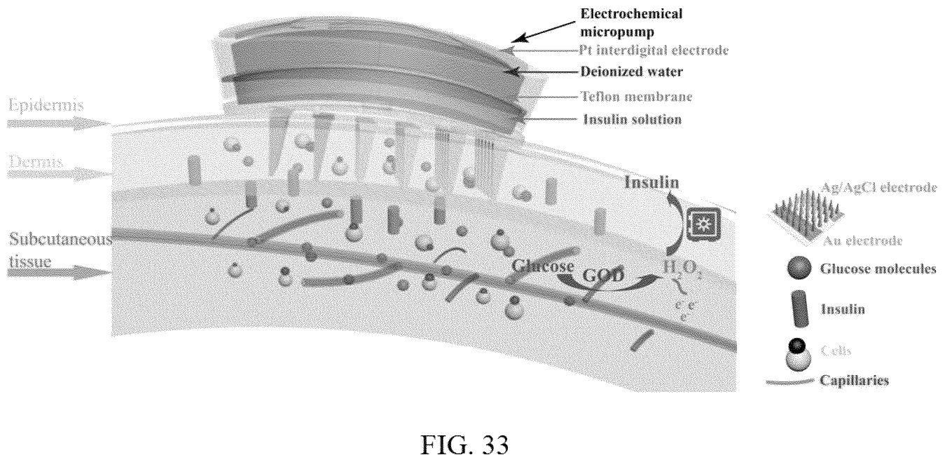

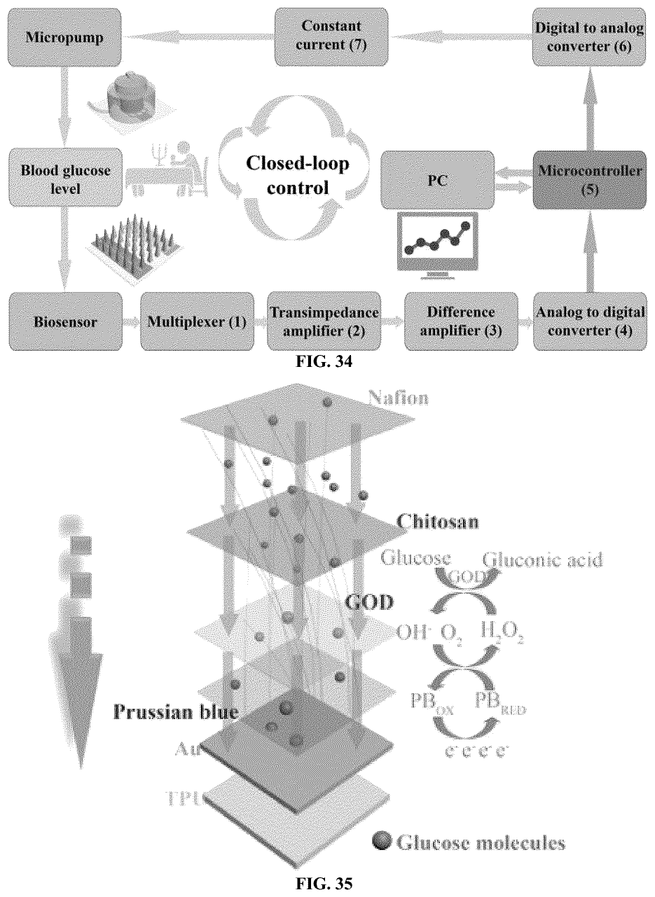

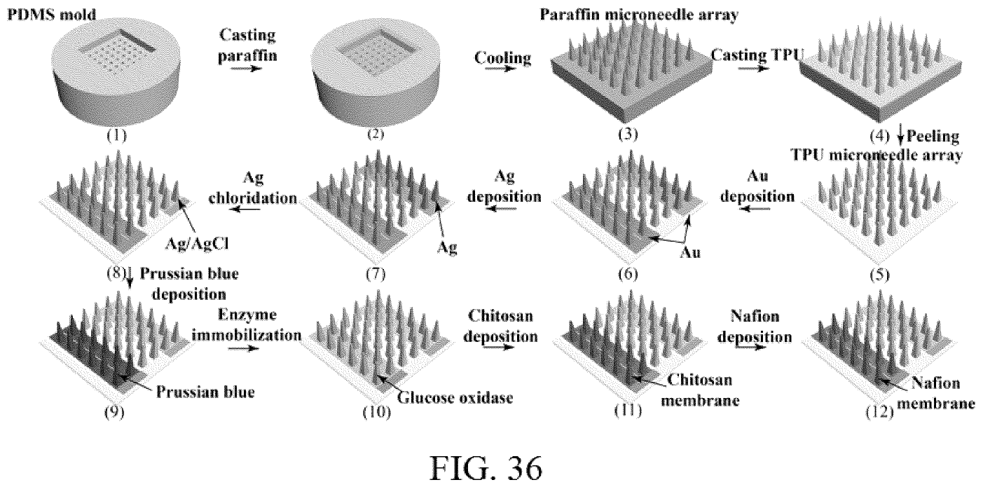

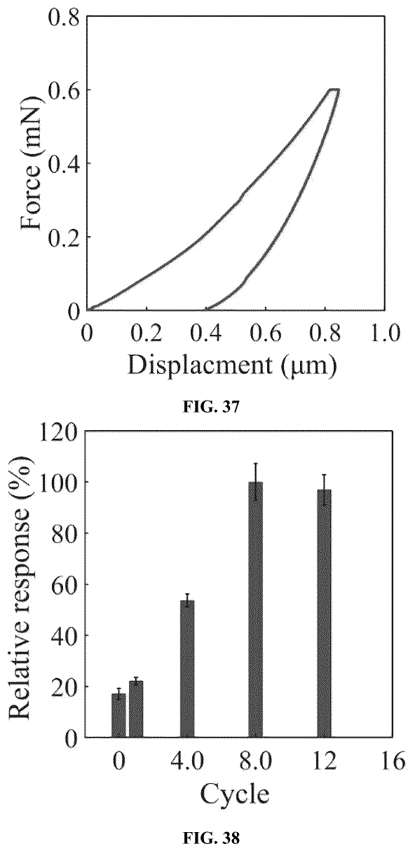

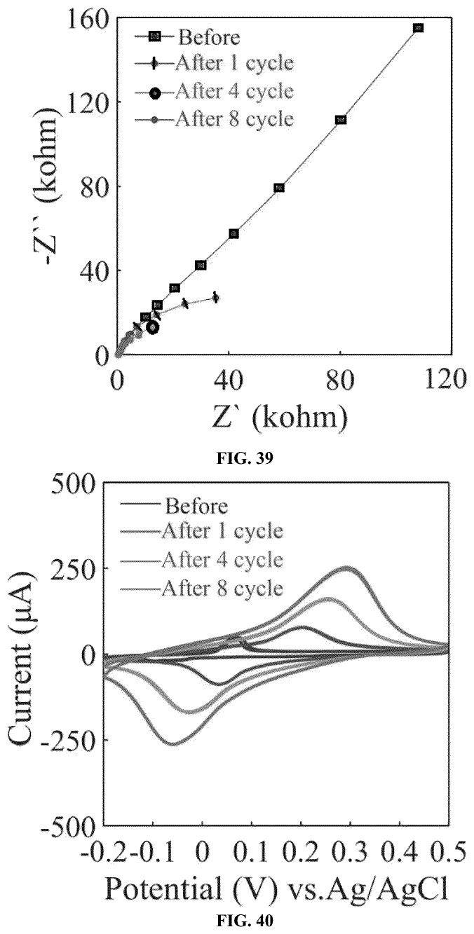

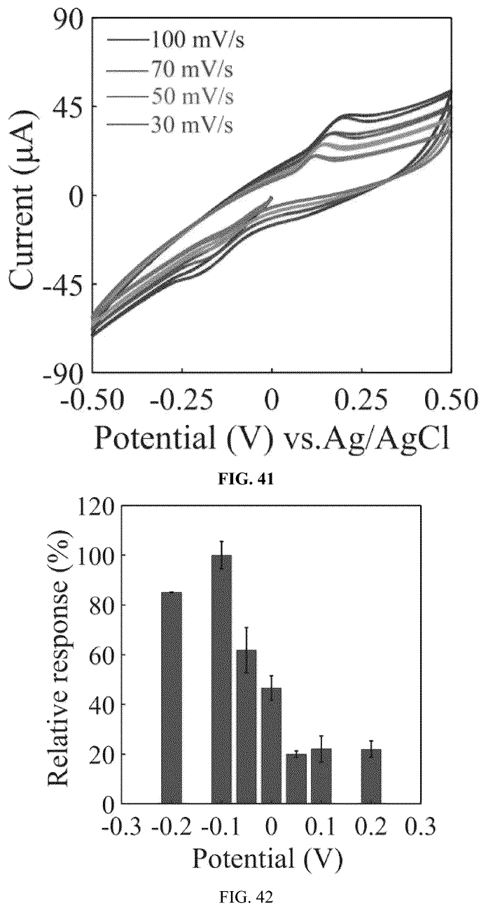

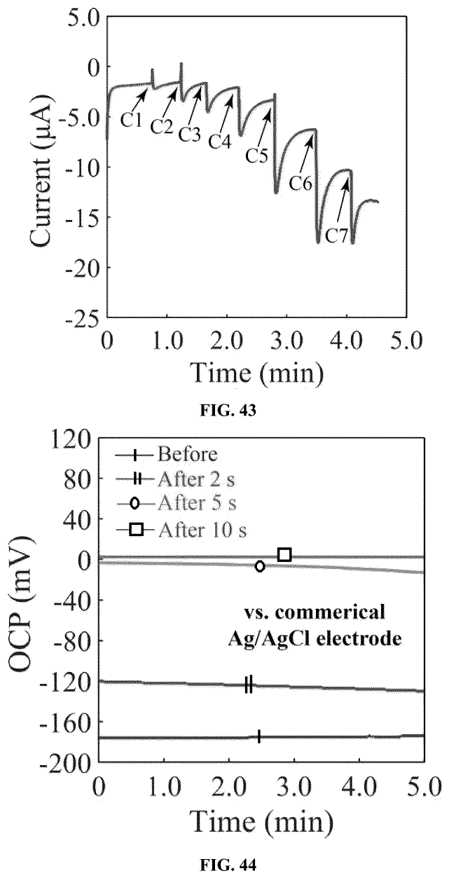

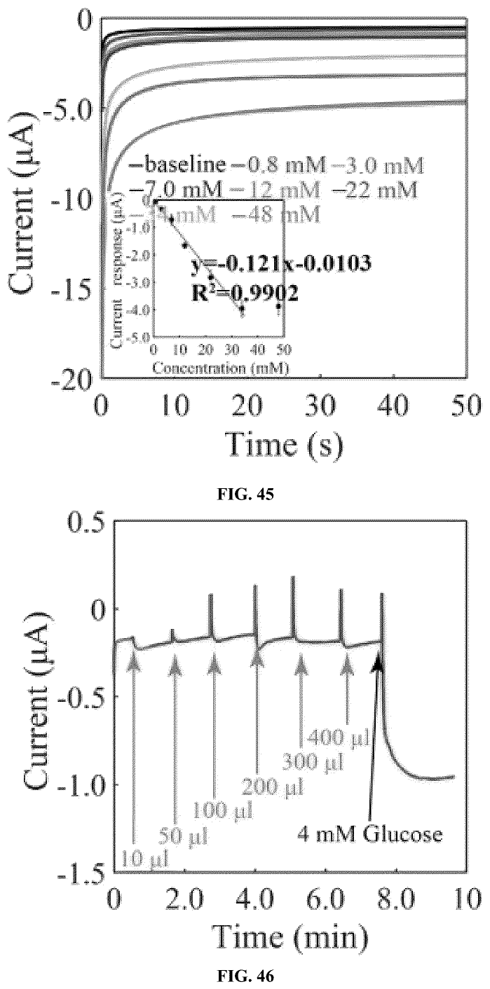

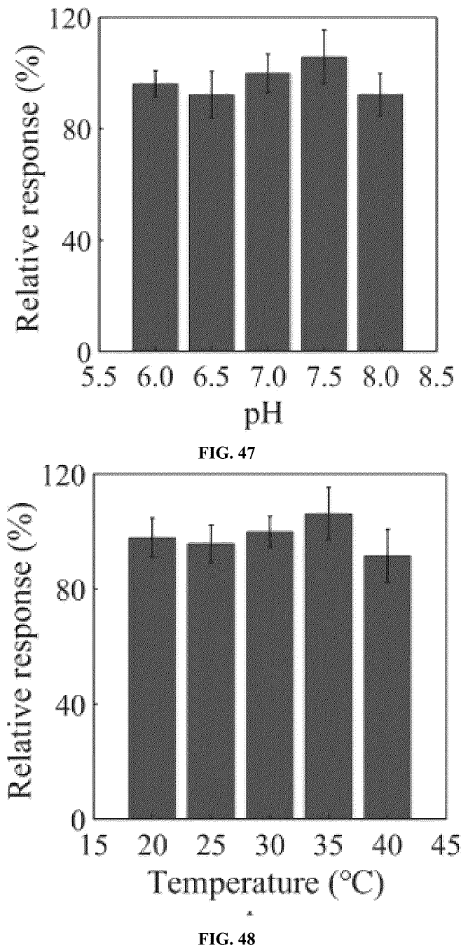

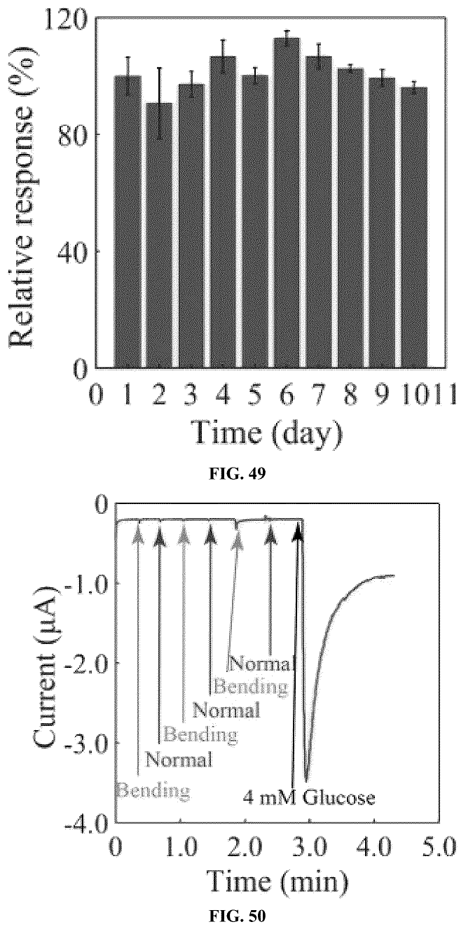

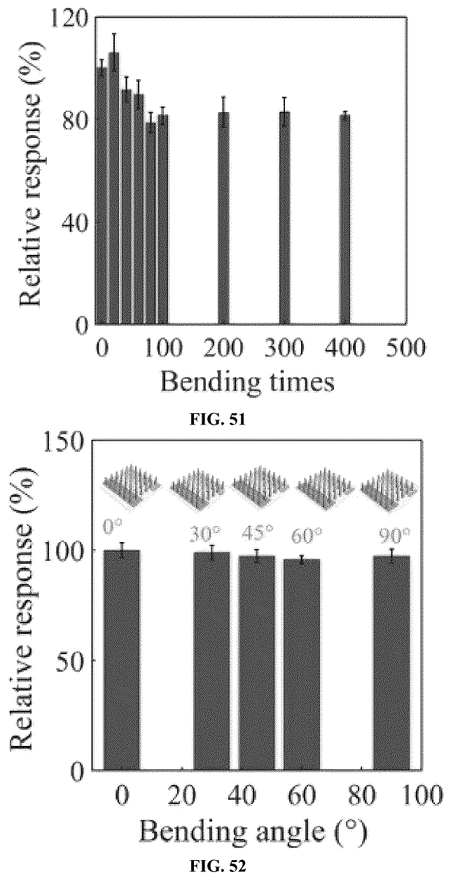

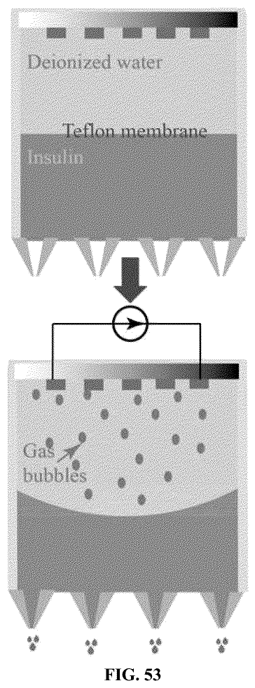

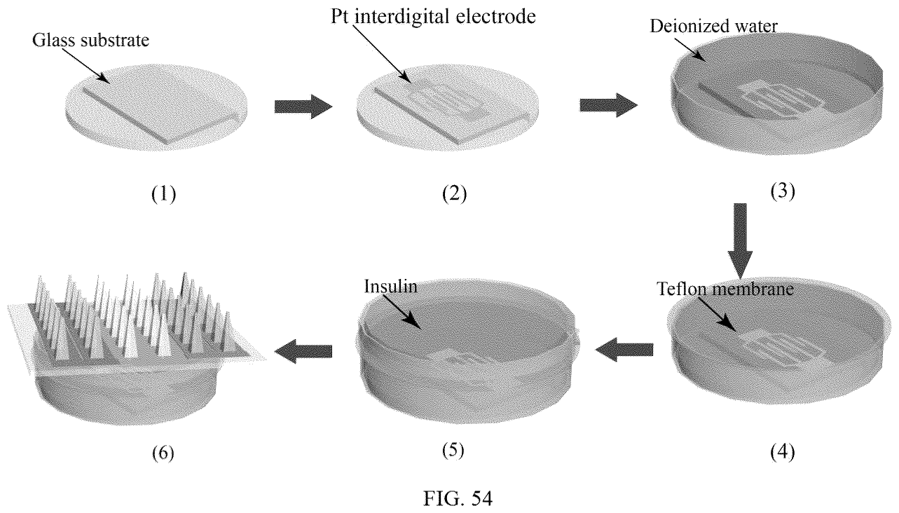

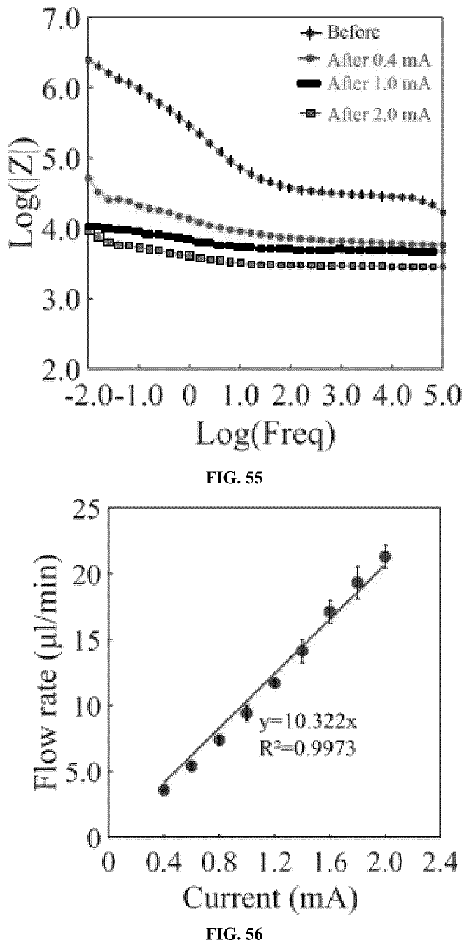

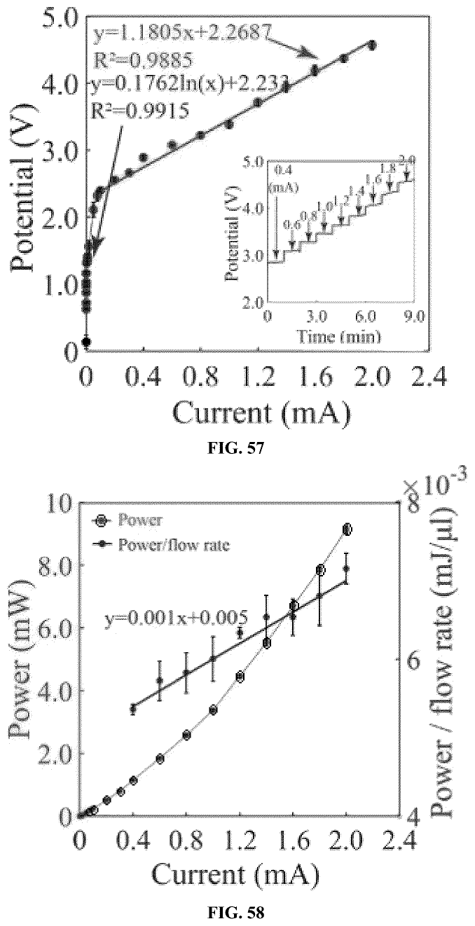

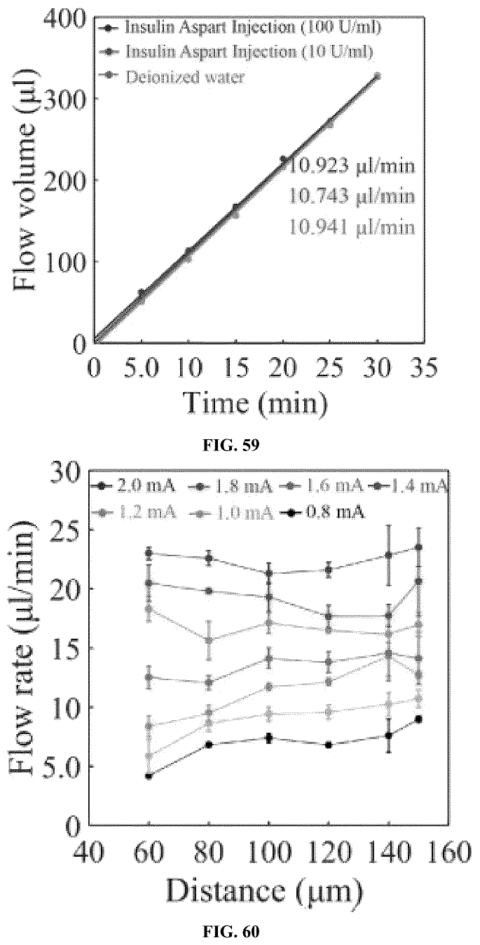

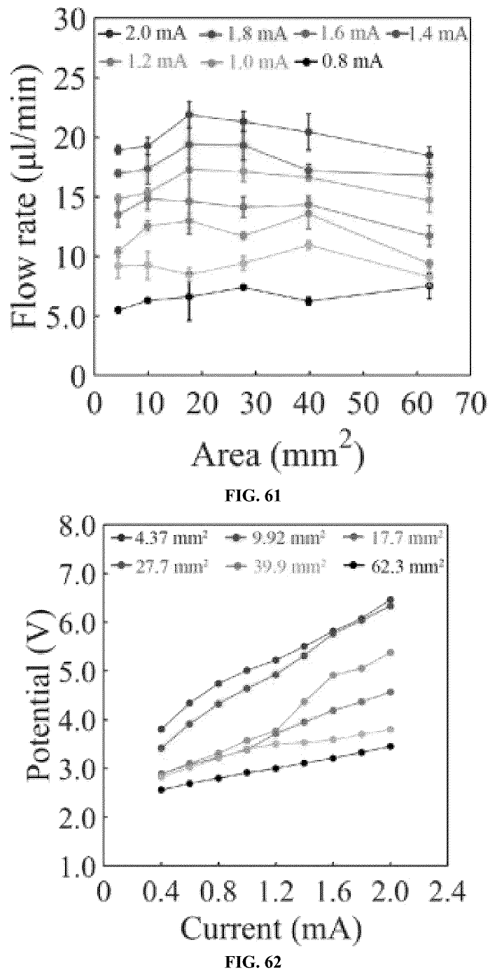

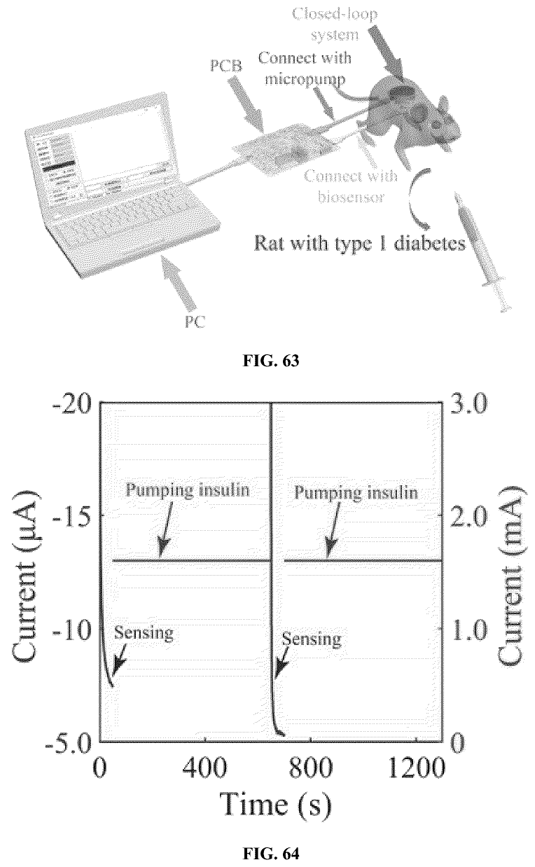

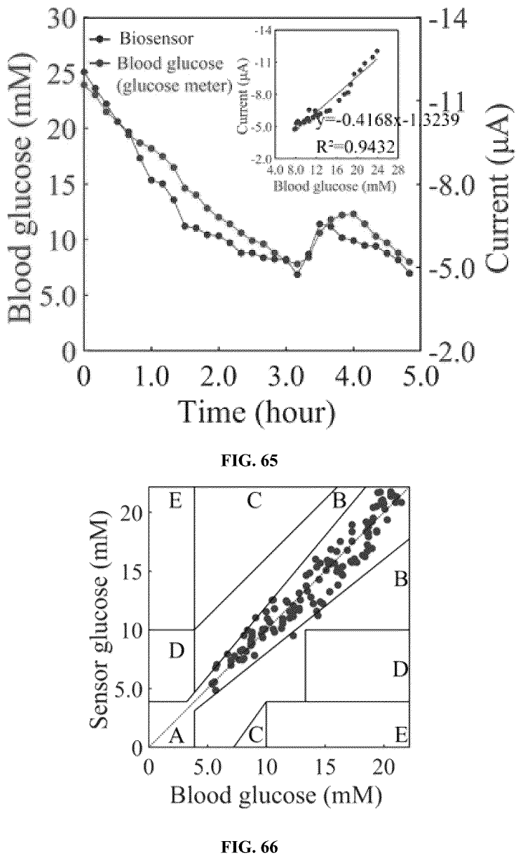

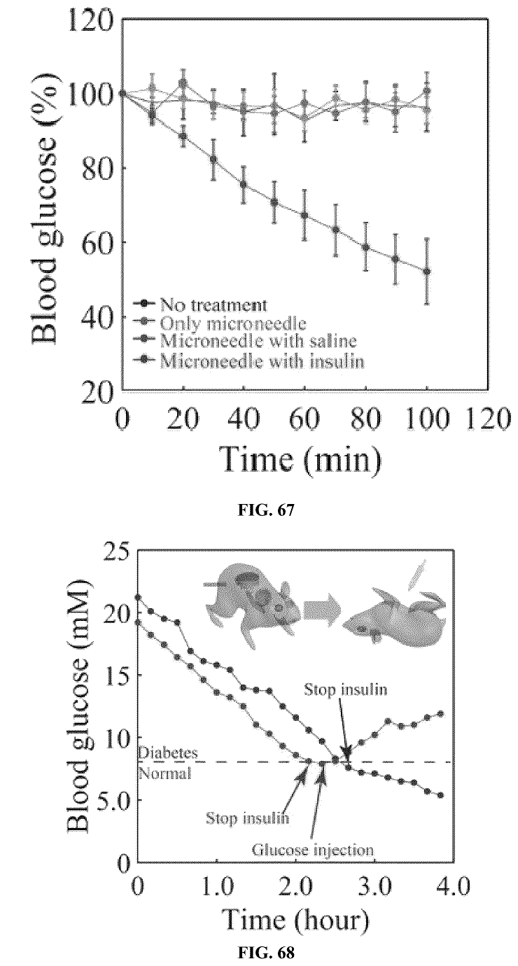

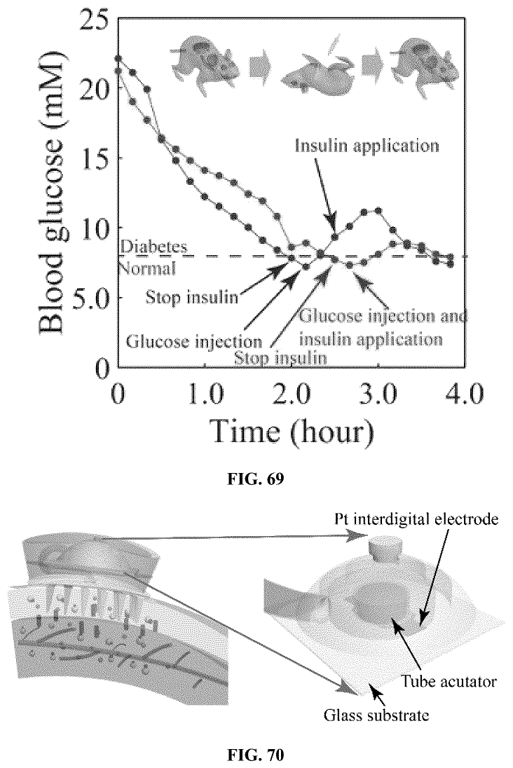

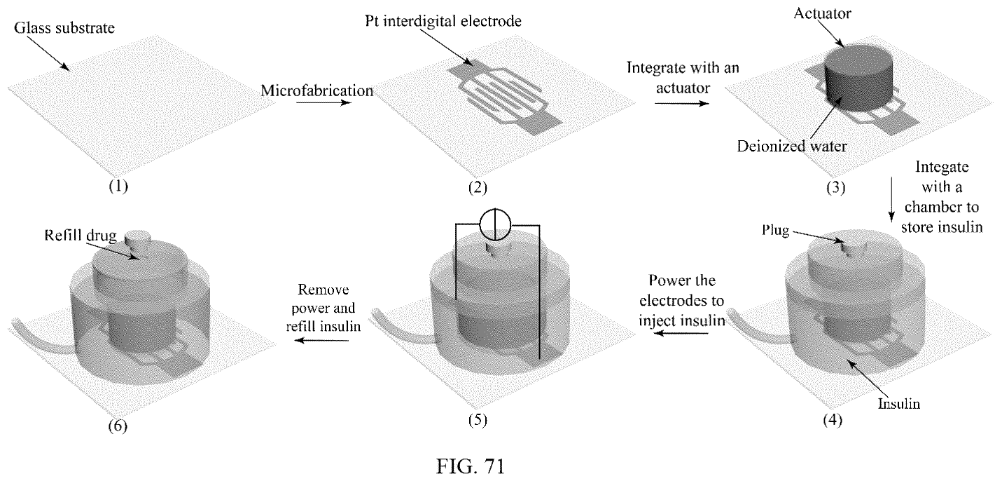

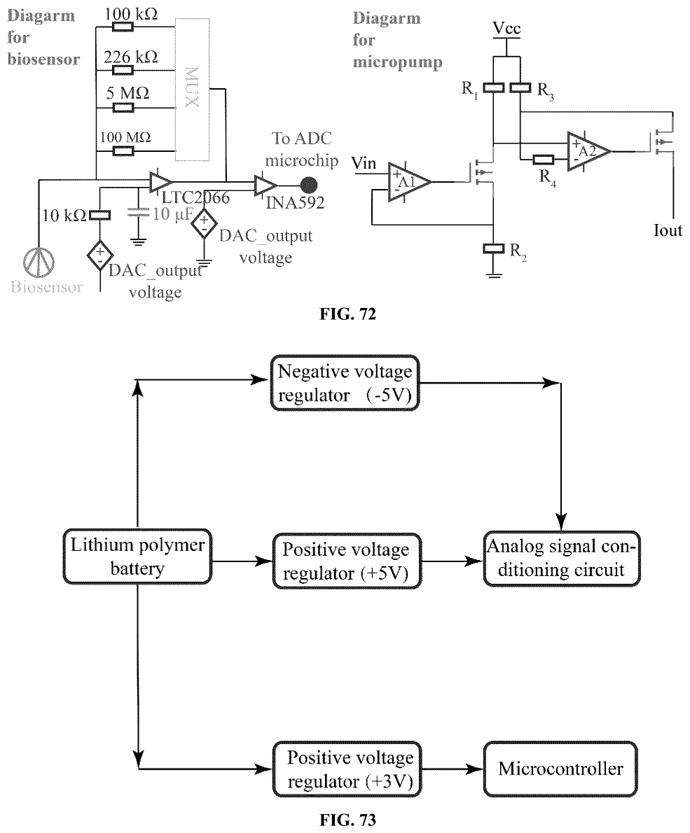

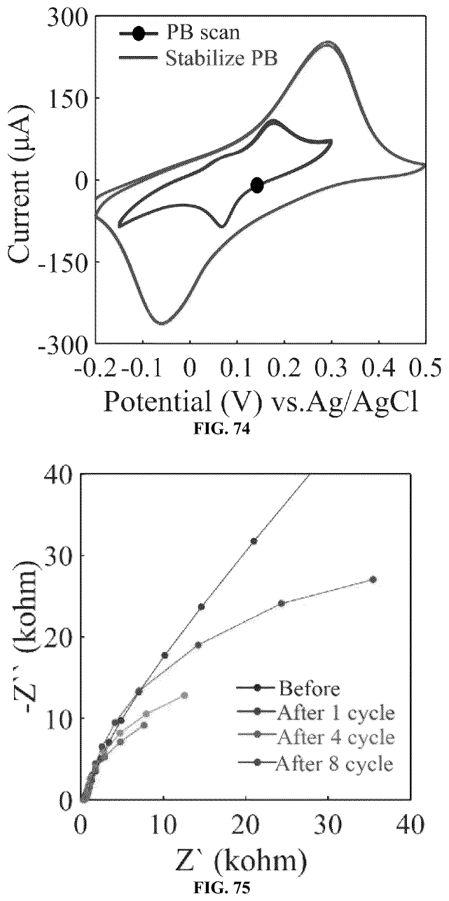

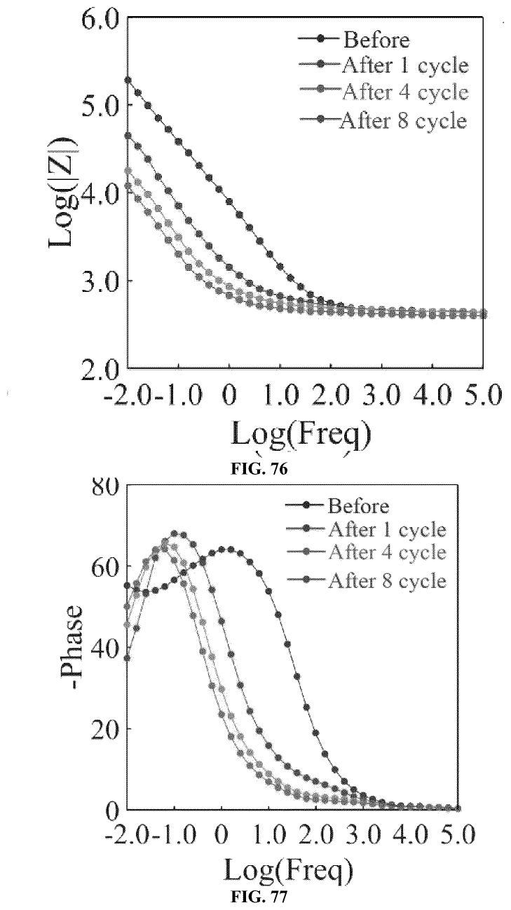

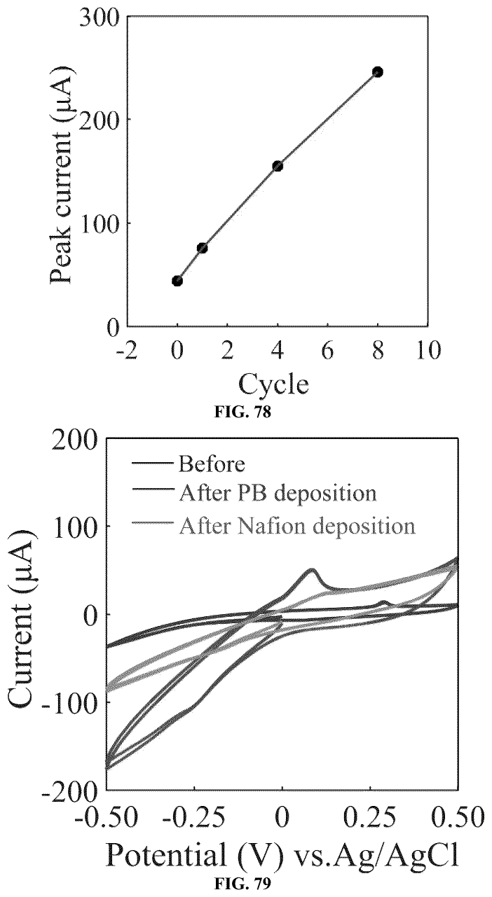

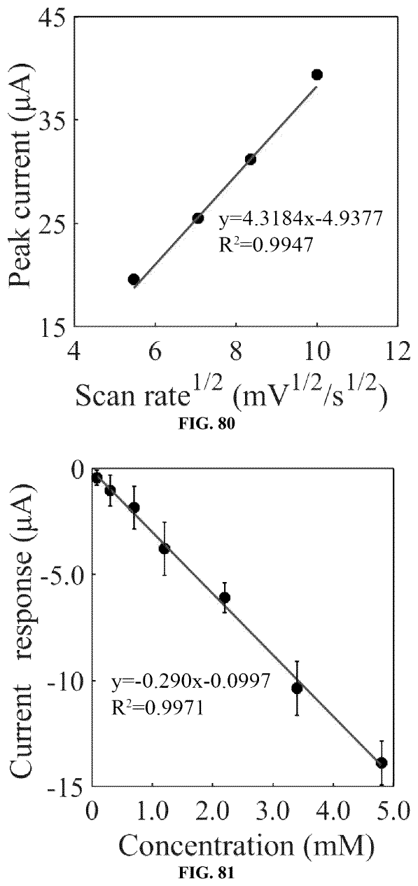

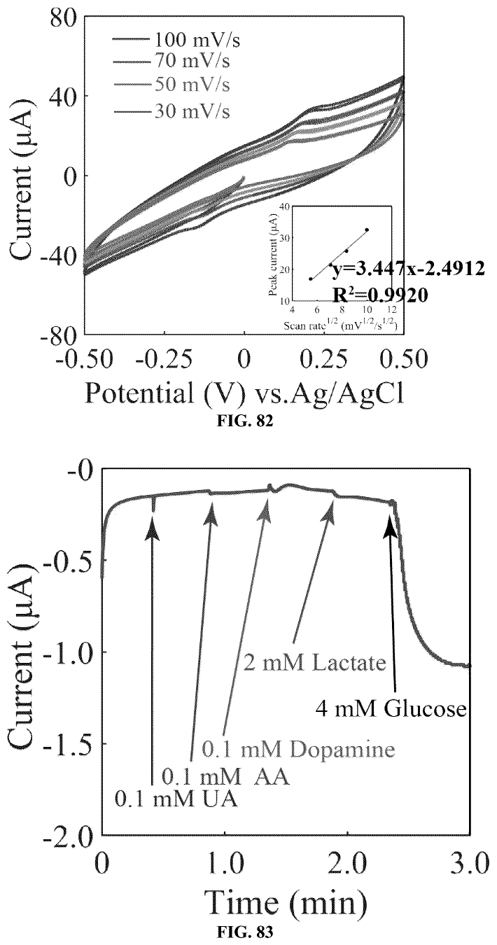

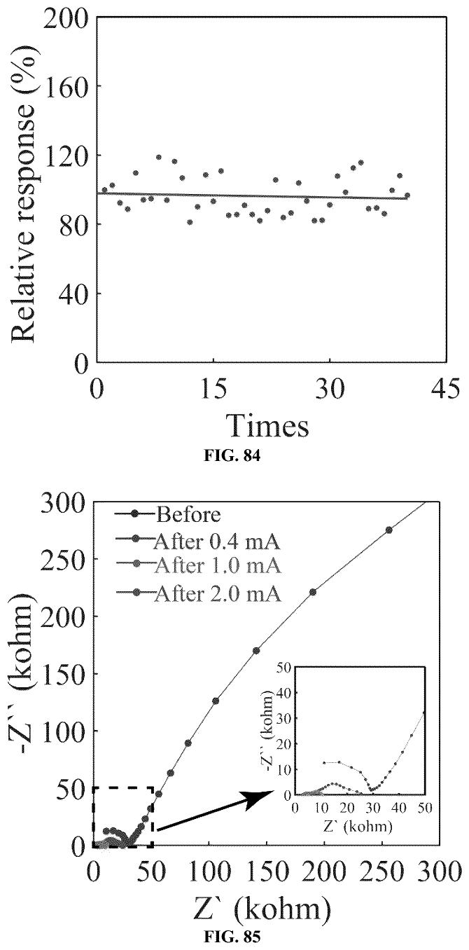

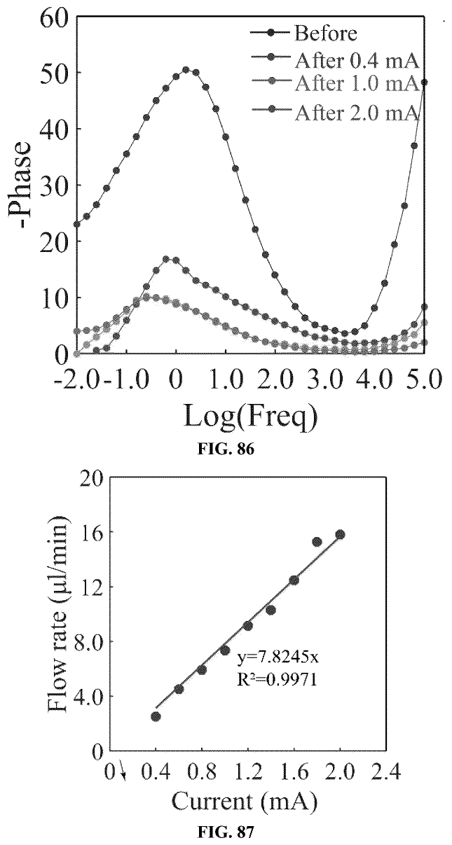

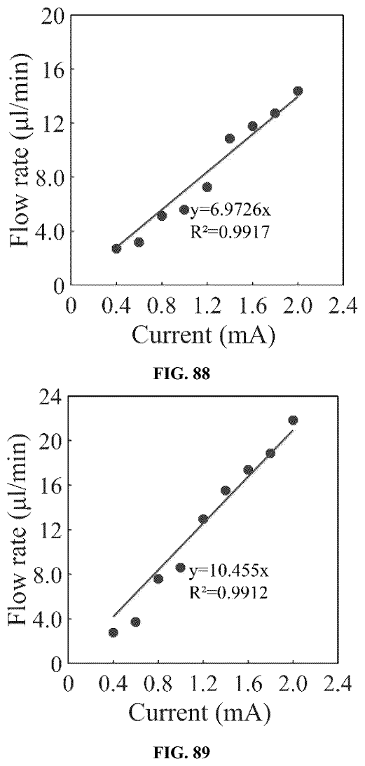

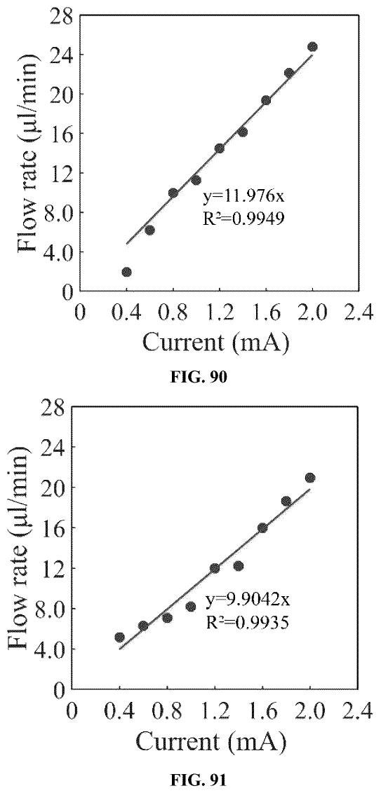

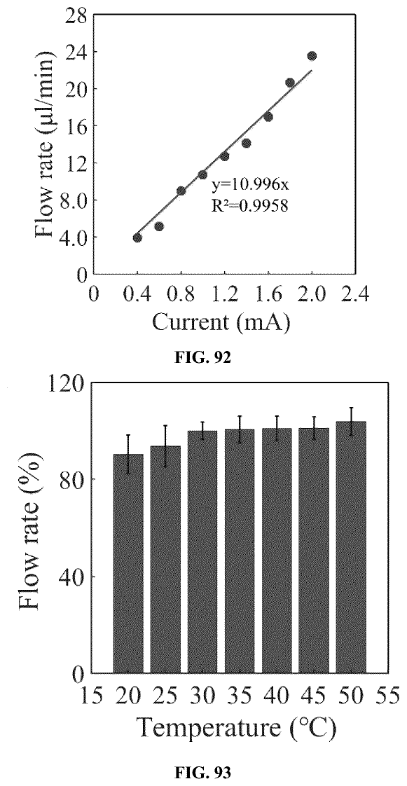

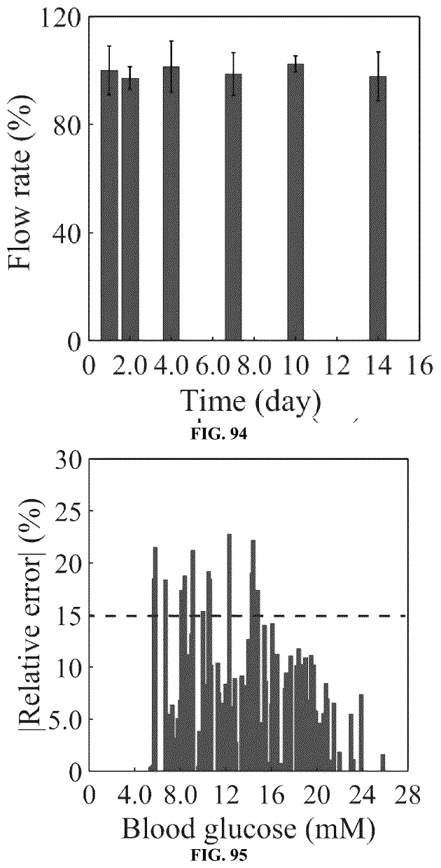

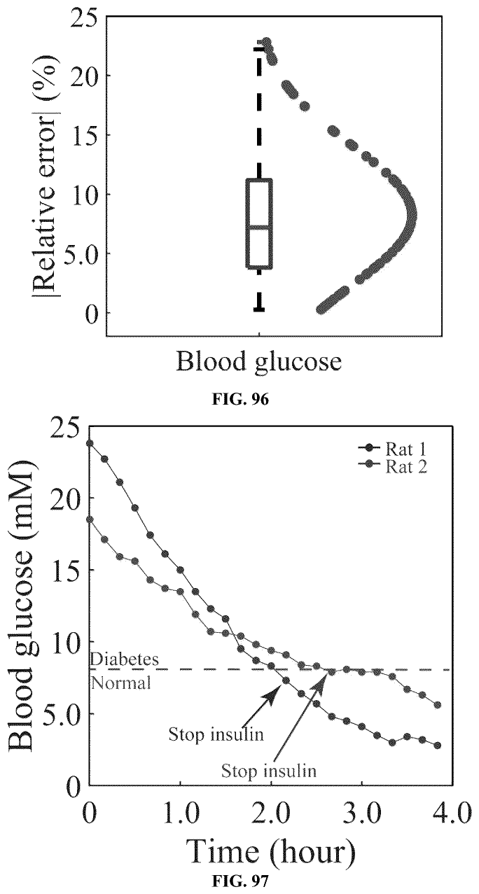

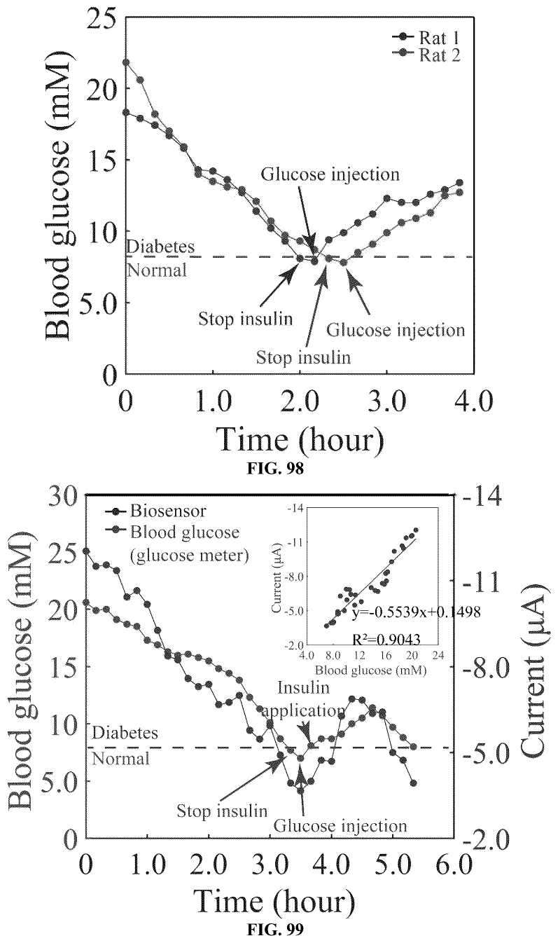

Now the present disclosure is described in combination with a specific application (see FIGS. 33 to 99).

Overall principle and structure of the feedback diabetes system.

The feedback system was constructed based on the flexible hollow TPU microneedles. The microneedles were placed on the skin and inserted into the dermis layer. Users would have little or no pain and bleeding by using the microneedles. The working and reference/counter electrodes for the microneedle sensing device were fabricated on the outer layer of the microneedles for the transdermal detection of dermal interstitial glucose. After constructing the sensor, a refillable electrochemical micropump was integrated with the TPU microneedles for the controlled release of insulin into the dermis layer via the hollow channels.

The entire closed-loop system was 2 cm in diameter and 1.2 cm in height, making it small, wearable, convenient and friendly to users. The working electrode was in Au with immobilized GOD (yellow color), and the reference/counter electrode is in Ag/AgCl (silver color). Each electrode, occupying two columns of microneedles, had a length of 1 cm and a width of 0.24 cm. The Pt interdigital electrodes were on a glass substrate with an overall dimension of 0.9 cm by 0.77 cm for electrolyzing deionized water to generate the gas bubbles. A stable sensor-skin interface can be formed due to the flexibility of the microneedle array. The closed-loop system was small in size, and it could be worn during daily activities without an obvious feeling to its existence. The system can be in a different shape with a larger volume to store insulin. A photographs of a PCB with the circuit flows for the sensor and the micropump to achieve a closed-loop function for the device is provided. The PCB had a dimension of 5.1 cm×5.1 cm, and was powered by a lithium-ion polymer battery with a voltage of 7.4 V (7 cm×6 cm×1.1 cm). In practical applications, the PCB would be further improved and re-designed by professional PCB and microprocessor engineers to reduce its size and make it more wearable.

A signal processing path from sensing interstitial glucose to powering the pump to inject insulin to achieve an automatic closed-loop diabetes management is provided. A PCB was operated via a multiplexer, a transimpedance amplifier, a differential amplifier, an analog-to-digital converter, a microcontroller, a digital-to-analog converter, and a constant current power source. Via these components, the PCB would power the sensor at a constant potential (0-0.3 V), process the sensing current signal to obtain a predicted blood glucose value, further apply a constant current (0-5 mA) to drive the micropump to deliver insulin, and transmit the data to the computer. After a short period of the insulin release, the biosensor would start to perform the sensing again. This sensing and pumping processes were repeated until that the blood glucose level reached a normal concentration. Users could choose the appropriate type and concentration of the insulin solution according to their initial blood glucose levels.

Fabrication of the Microneedle Biosensing Device

The biosensing device was constructed on the microneedles with a working electrode, and a reference/counter electrode. The working electrode possessed a multilayered structure to achieve an excellent sensing performance with a good biocompatibility. The working electrode was built on the outer surface of the TPU microneedles, and the layers of the working electrode included an Au thin film, a Prussian blue (PB) film, a GOD enzyme layer, a chitosan layer, and a Nafion membrane. A thin-film Ag/AgCl electrode was constructed as the counter/reference electrode. A PB layer was deposited as the electron transfer mediator to lower the working potential to −0.1 V and increase the sensitivity for detecting glucose. The GOD layer was used to catalyze glucose to generate H₂O₂ that was further mediated by Fe(CN)₆³⁻ in the PB film to produce the electrons, resulting in a current increase. The chitosan membrane was used as the encapsulation matrix for GOD, and it can also protect the enzyme from leakage. The antibiofouling and biocompatible Nafion membrane was used to eliminate the interferences and control the glucose diffusion selectively.

TPU is generally considered as an elastomer that is the bridge between rubber and plastics. It is a linear segmented copolymer composed of hard and soft segments separated by a microphase with great elasticity, flexibility, biocompatibility and high abrasion resistance. TPU is suitable for a variety of biomedical applications, such as manufacturing the medical catheter, heart assist devices, antibacterial coating, and wound dressing. Compared with other common medical materials (PE, PP, TPE, PVC, or silicone rubber), TPU elastomer has obvious advantages. For example, TPU has better mechanical and processing properties than silicone rubber, and it has a lower adsorption of drug, a better biostability and a higher biocompatibility than PVC.

The fabrication process of the TPU hollow microneedles was based on a soft lithography with the advantages of being convenient, cost-effective, non-toxic and easy-to-process. A PDMS mold with the negative patterns of microneedles made by a Boyue C8 laser cutting machine (Shichuangai Technology Co., Ltd., Hefei, China) was used to fabricate the hollow microneedles via soft lithography. The first step for constructing the sensor was to melt the paraffin in the PDMS mold, and the paraffin microneedle array was then formed after cooling and solidification. The TPU microneedle array with the hollow structure was obtained via casting the TPU solution on the paraffin microneedle mold, followed by drying and peeling off from the mold. After this, square holes can be formed on the bottom of TPU microneedles. However, some tips of the microneedles may be blocked, and a stainless steel needle (100 μm in diameter) can be further used to penetrate the microneedle tips to obtain clear holes. Further, the Au and Ag thin-film sensing electrodes were constructed by a physical vapor deposition (PVD). The Ag electrode was then chloridized to form the Ag/AgCl layer as the reference/counter electrode. The Au electrode was modified successively by a Prussian blue layer, a GOD matrix, a chitosan layer, and a Nafion membrane to form the working electrode. Each pyramid microneedle had a height of 1.5 mm and a square base dimension of 0.4 mm. A square hole can be seen at the tip of the microneedle with a dimension of 100 μm, and the hole on the bottom of the microneedle had a dimension of 340 μm. The thickness of the microneedle sidewall was measured to be 45 μm. The shape of the microneedle after being inserted into the skin was almost unchanged, indicating an excellent mechanical stability of the microneedles.

Refillable Electrochemical Micropump

The electrochemical micropump is based on generating the gas bubbles of hydrogen and oxygen through the electrolysis of water, by using the interdigital electrodes in platinum, to further drive insulin into the inner channels of the microneedles. When there is no electrical power provided to the micropump, the insulin solution would not leak from the holes of the microneedles due to the capillary force. When a constant current is applied to the electrodes in the micropump, deionized water would be electrolyzed to generate the hydrogen and oxygen gas bubbles. The Teflon membrane has a great flexibility and can easily achieve a deflection. The Teflon membrane is initially flat and would be inflated by the pressure exerted by the growing gas bubbles. The smooth deformation of the Teflon membrane would drive the insulin solution to release from the hollow microneedle array. When the power is removed, the hydrogen and oxygen gases could recombine into water via the catalysis of Pt interdigital electrodes, accompanied by the shrinking of the Teflon membrane and the decrease of the actuator size. The generation of gases and their recombination are controlled by the Pt interdigital electrodes, which allow the turning on and off the pumping for multiple cycles, and it is especially suitable for repeatable uses. In addition, the Teflon membrane prevents insulin from contacting the electrolyzed fluid in the actuator and avoids any possible oxidation or reduction of the insulin solution.

Pt is known as the most suitable material for water electrolysis and can catalyze the recombination of bubbles on the electrode surface to form water. The design of the interdigitated electrode could reduce the resistive path through the electrolyte and improve efficiency. The Pt interdigital electrodes are first patterned via photolithography and physical vapor deposition on a glass slide. On the electrode, there is a small chamber filled with deionized water as the electrolyte, and then it is adhered with a Teflon membrane to be the actuator. Insulin is then placed on the Teflon membrane. The hollow microneedle arrays are further placed on the insulin solution. The micropump can provide a repeatable and controllable delivery of insulin with a simple fabrication procedure.

The area for all the fingers with a Pt deposition was 27.7 mm². Each finger had a width of 0.1 mm, and the distance between two adjacent fingers was also 0.1 mm. The surface of the original Pt electrode was smooth, while after working at 3 mA for 30 min, some areas of the Pt electrodes were damaged due to the oxidation and degradation by the current, and the EDS analysis shows an obvious O element peak after the electrode was oxidized and damaged. The damage to the Pt electrode could be reduced by lowering the applied current value. The Pt electrodes can be relatively stable at a current of 2.0 mA for nearly 12 h. There was only a slight damage to the edge of the Pt electrode after working for 12 h continuously, and the little damage had almost no effect on pumping insulin.

A 30 μm-thick Teflon membrane was used to cover the Pt interdigital electrodes to function as the actuator that was further integrated with the 3D-printed chamber. Before working, the actuator had a diameter of 6.5 mm and a height of 8 mm, containing 265 μl of deionized water. After working for 10 min at 1 mA, the volume of the actuator was expanded to about 350 μl with a diameter of 7 mm and a height of 9.2 mm. After working for 20 min at 1 mA, the size of the actuator was expanded with a diameter of 7.5 mm, a height of 10.5 mm, and a volume of 450 μl. After removing the power for 20 min, the actuator almost returned to its original volume.

Compared to other micropumps used for closed-loop diabetes management, this micropump shows clear advantages in terms of small size, high insulin delivery efficiency, availability for delivering different concentrations of insulin, and excellent stability of use.

Evaluation of the Closed-Loop System on Diabetic Rats

Diabetic SD rats were selected as the experimental objects for the in-vivo evaluation of the closed-loop feedback system. The rat had been induced to be the subjects with type 1 diabetes by injecting STZ according to the protocol. The closed-loop system was fixed on the rat's abdomen, and powered and controlled by the PCB that could be interacted with the computer. The skin irritation experiment was conducted for evaluating the biocompatibility of the microneedles on the rats' skins. The results showed that there was no obvious erythema or edema on the rats' skins before and after application of the system for 24 h, 48 h and 72 h, which demonstrated that the microneedle biosensing device had excellent biocompatibility.

To reduce the frequency of adding insulin into the micropump and evaluate the long-term performance of the system, the in-vivo experiments were conducted with the system. To achieve a closed-loop control of the blood glucose levels in diabetic rats, a two-step model was adopted with a sensing time of 50 s and a pumping time of 10 min alternatively. After a 50 s′ glucose measurement by the biosensor, the final current value in the i-t curve was recorded and sent to the microprocessor. The final current in the first blue line was −7.43 μA, corresponding to a blood glucose level of 16.3 mM. When the current was higher than the critical value, the micropump was then driven by a constant current (1.6 mA) to inject insulin (30 U/ml) for 10 min continuously, then the biosensor performed the test of blood glucose again. The final current in the second blue line was −5.29 μA and the blood glucose was decreased to be 8.1 mM. The alternative sensing and pumping were repeated until the blood glucose reached the critical value. After that, the pumping of insulin was stopped, and only the glucose measurement was conducted.

To obtain an automatic and effective closed-loop management of diabetes, the first step was to establish the correlation between the current value from the biosensor and the blood glucose level from the clinically approved glucose meter. The current change measured by the biosensor matched well with the glucose meter results. An obvious linear relationship between the current change and blood glucose level change was obtained with a slope of 0.4168 μA/mM and R2 of 0.9432. This result proved that the biosensor could reliably respond to the fluctuations in blood glucose. The Clark error grid was employed to study the difference between the blood glucose levels measured by the microneedle sensing device and the commercial glucometer (the data was from six rats). All 137 points were positioned in the clinically acceptable error zone A and B. The Clark error grid was better compared to our previously reported work, which may be due to the longer height of each microneedle (1.5 mm) compared to the previous one (1.0 mm) and the deeper insertion channel formed in the skin with a larger contact area between the interstitial fluid and each microneedle. In addition, the structure of the working electrode (PB modified Au electrode with deposition of enzyme layer, chitosan layer and Nafion layer) and microneedle material of the biosensor was different from the previous one, and these all had an impact on the sensing accuracy. The mean absolute relative difference (MARD) value between these two detection approaches was calculated to be 8.215%±5.907%, and the error of the biosensor was from 0.274% to 22.8%, and 75% of points were lower than 11.19%. The results fulfilled the ISO15197:2013 accuracy limits criteria, and demonstrated that the microneedle biosensing device had high accuracy for determining the blood glucose levels.

Without the application of the microneedle device, the blood glucose was not lowered. When the microneedle device was applied on the skin without the injection of any liquid or with the injection of saline, the blood glucose was not lowered as well. The results demonstrate that the microneedle device itself had no effect on blood glucose. When the microneedle device was applied to the skin with the delivery of insulin into the inner channels by the electrochemical micropump, the blood glucose level sharply declined to about 50% of its initial value in 100 min (from six rats). These results demonstrated that insulin can be injected into interstitial fluid effectively with the closed-loop system based on the hollow microneedles and exerted a significant antihyperglycemic effect on diabetic rats.

The injection of glucose was to simulate the food intake to increase the blood glucose levels. The closed-loop systems were applied to the rats for about 2 h at the beginning, and the blood glucose levels decreased dramatically, when the glucose levels reached the normal levels, the closed-loop system stopped the injection of insulin automatically. After that, two conditions were compared, one condition was without any operation, and the other condition was injecting glucose intraperitoneally. Without any operation, the blood glucose levels of the diabetic rats kept decreasing for another short period of about 1 h and maintained within the normal glucose range (from three rats). While for another condition with the injection of glucose (0.1 g/kg) intraperitoneally, the blood glucose levels increased shortly to be higher than the normal glucose range (the data was also from three rats). This may be because that without insulin, when the diabetic rats were injected intraperitoneally with a large amount of glucose at one time, the glucose molecules were absorbed into the blood in a short time, causing a rise in blood glucose.

Two situations were studied for the closed-loop system operated with an alternative sensing time of 50 s and pumping time of 10 min. In the first situation, by operating the closed-loop system to sense glucose levels and inject insulin for about 2 h from the beginning, the blood glucose level decreased dramatically. At the time of 120 min, when the blood glucose level decreased from 22.1 mM to 7.8 mM, lower than the critical glucose level (8.3 mM), the closed-loop system sensed the current value from the biosensing device, and stopped the injection of insulin automatically. The blood glucose kept decreasing due to the effect of the previously delivered insulin. After 10 min, the blood glucose level decreased to 7.2 mM, glucose was injected intraperitoneally, and the blood glucose level increased shortly. When the blood glucose level reached a concentration above the critical value (8.3 mM) and when the closed-loop system was in the sensing mode, the system would sense the current value from the biosensing device and start the injection of insulin automatically. At the time of 140 min, the blood glucose level increased to 8.0 mM (lower than 8.3 mM) and no insulin was injected. At the time of 150 min, the blood glucose level increased to 9.3 mM (higher than 8.3 mM), and the closed-loop system began to inject insulin. At the beginning of this injection of insulin, the blood glucose still kept increasing due to the small amount of insulin. Gradually, as the increase of the amount of the injected insulin, the blood glucose reached the peak of 11.2 mM at the time of 180 min, and after that, the blood glucose began to decrease until it reached the critical value (8.3 mM) at the time of 220 min.

The second situation was shown as follows. With the operation of the closed-loop system by alternative glucose sensing and insulin injection from the beginning to the time of 150 min, the level of blood glucose kept decreasing from 21.2 mM to 7.7 mM that was a concentration lower than the critical concentration of 8.3 mM. The closed-loop device sensed the value to further stopped the injection of insulin automatically. After 10 mins, at the time of 160 mins, when the blood glucose level decreased to 7.3 mM, glucose was injected intraperitoneally. At the same time, the parameter in the software of PCB was adjusted to change its critical value to be 7.3 mM. Therefore, at this time with a blood glucose level of 7.3 mM, an automatic injection of insulin was turned on, which was earlier than that in the blue line. The blood glucose level began to increase, reached a peak value of 8.9 mM at the time of 200 min, and quickly decreased to the real critical value (8.3 mM) at the time of 220 min.

Compared to the first situation, the blood glucose fluctuation range in the second situation was smaller, and it could return to the normal level faster after the glucose injection. That may be because that the intake of insulin was earlier, accelerating the decomposition of the injected glucose in blood since the critical value was adjusted to the blood level when glucose was injected. It indicated that users could freely set the time when the insulin starts to be released according to the actual situation and prevent a sharp increase in blood glucose after the glucose intake. The function of the system in different diabetic rats may be variable due to their different sensitivities to insulin. It was often reflected in the decline rate of blood glucose levels. For example, for terminal-stage diabetic rats (the modeling time was more than one month), this system also performed well for the management of blood glucose, though it needed a longer time to decrease from a high blood glucose level to a normal level. All these results proved that the system could achieve an automatic closed-loop control of blood glucose in diabetic rats successfully.

### Experimental

Construction of the TPU Microneedle Array

A 35% (wt %) medical grade thermoplastic polyurethane (TPU) solution was obtained by mixing the TPU powders with dimethylformamide with heating at 60° C. for 1 h. To fabricate the TPU hollow microneedles, the paraffin microneedle mold was firstly obtained by melting the paraffin at 160° C. and coating the melted paraffin onto a PDMS mold (from Laike Mould Co., Suzhou, China), followed by a peeling off. The TPU solution was then evenly coated on a paraffin mold and cured at 40° C. for 24-48 h. The hollow TPU microneedle array was obtained by a peeling off from the paraffin mold. The microneedle array was constructed in a 6×6 array. Each microneedle was in a pyramid shape with a bottom width of 340 μm and a height of 1.5 mm. The distance between each microneedle was 2 mm.

Construction of the Biosensing Device

The sensing electrodes were constructed on the TPU microneedle array with an Au working electrode and an Ag/AgCl counter/reference electrode. Each electrode has a length of 1 cm and a width of 0.24 cm, occupying two rows of microneedles with a distance of two rows in between. The working electrode was fabricated by depositing Au/Ti (200 nm/20 nm) onto two rows of the microneedles. For the Ag/AgCl electrode, a layer of Ag in 200 nm thickness was firstly deposited on the Ti—Au electrode, followed by immersing the Ag electrode into 50 mM ferric chloride (FeCl₃) solution for 10 s. Then, to remove the impurities and activate the Au working electrode, the microneedle array was immersed in 0.1 M H2SO4 solution for a CV scanning for 20 cycles) potential range: 0.2 V to 1.2 V; scanning rate: 1 V/s). To deposit the PB layer onto the Au electrode, the microneedle array was immersed into a freshly prepared solution containing 2.5 mM FeCl₃, 100 mM KCl, 2.5 mM K₃Fe(CN)₆ and 100 mM HCl for a CV scanning for 8 cycles (potential range: −0.15 V to 0.3 V; scanning rate: 20 mV/s). Finally, the microneedle array was immersed into the 0.1 M KCl/HCl solution for a CV scanning from −0.2 V to 0.5 V at a scanning rate of 50 mV/s for 4 cycles in order to stabilize the PB layer.

Before immobilizing GOD on the working electrode, a UV ozone cleaning was performed on the sensor for 10 min to obtain a hydrophilic surface. Then, a 5 μl of GOD (50 U/μl) solution was mixed with a 5 μl of bovine serum albumin (BSA) solution (1%) and a 10 μl of diluted glutaraldehyde (2%) solution, and the mixture was coated on the Au working electrode. The sensor was dried at 4° C. for 30-60 min, and then a 10 μl of 1% (wt %) chitosan solution that was dissolved in the 2% (wt %) acetic acid was deposited on the Au electrode. After drying for another 2-4 h, a 10 μl of Nafion (0.5%, (wt %)) solution was coated on the working electrode. The sensor was finally stored in the refrigerator (4° C.) overnight.

Construction and Characterization of Electrochemical Micropump

The Pt interdigital electrodes were patterned on a glass slide via photolithography (using the AR-P 5350 positive photoresist), sputtering, and a lift-off process. The electrodes had a dimension of 0.9 cm by 0.77 cm, a Pt/Ti layer thickness of 200 nm/20 nm, a finger width of 100 μm and a gap of 100 μm between two adjacent fingers.

To construct the electrochemical micropump, the 3D-printed hollow cylinder shells with a diameter of 2 cm were constructed and assembled with the Pt interdigital electrodes. A 30 μm-thick Teflon membrane was sealed with a 8 mm-high hollow cylinder shell and the Pt electrodes to contain deionized water as the electrolyte. On the Teflon membrane, another 3 mm-high hollow cylinder shell was placed to contain the insulin solution that was further assembled with the microneedle array. Other shapes of the pumps can be fabricated as well.

Design of the PCB

A PCB was designed to operate the closed-loop system, composed of the circuits to control the sensor and the pump. The core of this system was the stm32f103 microcontroller (MCU), the AD7171 analog to digital converter (ADC), the AD5541 digital to analog converter and the tmux6104 multiplexer (MUX). The MUX was functioned to select the current measurement range and accuracy with the assistance of four resistors (if the current was lower than 0.027 μA, select the 100 MΩ path; If the current was in 0.027-0.54 μA, select the 5 MΩ path; if the current was in 0.54-11.9 μA, select the 226 kΩ path; if the current was higher than 0.54-11.9 μA, select the 10 kΩ path.) The 10 KΩ resistor and 10 uF capacitor were used to remove the noise of the high-frequency output voltage. The voltage signal was then converted into the digital signal by the ADC and sent to the MCU. Then the MCU would send the current value to the computer, displayed on the interface. Users could save the data to perform further processing.

When the signal was higher than the critical value, the MCU sent the instructions, and the DAC translated instructions into the analog signal to drive the constant current power source to provide a constant current to the micropump. Iout=Vim/R2×R1/R3. When the resistors of R1, R2, and R3 were determined, the output current Iout of the circuit was only dependent on the input voltage Vin. As long as the Vin remained constant, the output current Iout was constant.

The PCB was powered by a lithium-ion polymer battery with a voltage of 7.4 V. A constant regulated output of +3 V was provided for the microcontroller and +5 V for the analog signal conditioning circuit. The negative power supply (−5 V) was also used for the analog signal conditioning circuit. The PCB was connected with a PC via a USB cable and exchanged data through a UART. Users could set the potential (0.1-0.3 V) being applied to the biosensor and the constant current value (0-5 mA) being supplied to the micropump, and the critical current value on this interface. The current measured by the biosensor was displayed in the display window continuously.

While the embodiments of the present disclosure have been described above with reference to the accompanying drawings, the present disclosure is not limited to the foregoing specific embodiments, and the foregoing specific implementations are merely illustrative rather than restrictive. Under the inspiration of the present disclosure, many other forms can be made by those of ordinary skill in the art without departing from the spirit of the present disclosure and the scope protected by the claims, which are all within the protection of the present disclosure.

What is claimed is:

1. A drug injection device based on an electrochemical reaction, wherein the drug injection device based on the electrochemical reaction is used to generate a driving force based on electrochemical reaction, and to automatically drive drug solution under the driving force to administer a drug to a patient.

2. The drug injection device based on an electrochemical reaction according to claim 1, wherein the drug injection device based on the electrochemical reaction is a drug injection pump based on electrochemical reaction, and the drug injection pump comprises a driving component and a drug storage component, wherein:
the driving component is arranged inside the drug storage component, and is used to generate a driving force based on an electrochemical reaction principle, the driving force is applied inside the drug storage component, the driving component comprises an electrochemical element which is connected to the outside of the drug storage component via a wire and is used for receiving a preset current; the electrochemical element is used for generating a gas based on the preset current, the gas is used for generating the driving force, and the electrochemical element is an electrode with a nano or micron thickness fabricated by a metal evaporation process, a screen-printing process or a magnetron sputtering process;
the drug storage component is internally loaded with drug solution, and the drug solution is pushed to the outside of the drug storage component along at least one liquid outlet hole on the drug storage component under the driving force, thus administering a drug to a patient through the liquid outlet hole, or administering the drug solution to a patient along an injection mechanism connected to the liquid outlet hole.

3. The drug injection device based on an electrochemical reaction according to claim 2, wherein the electrode is a metal electrode, a carbon electrode, or a composite conductive material electrode.

4. The drug injection device based on an electrochemical reaction according to claim 3, wherein the electrode is an interdigital electrode, a plate electrode, a pillar electrode or an irregularly shaped electrode.

5. The drug injection device based on an electrochemical reaction according to claim 4, wherein a substrate of the interdigital electrode is a hard substrate, a flexible substrate or a stretchable elastic substrate.

6. The drug injection device based on an electrochemical reaction according to claim 5, wherein the shape of the substrate is curved, planar, serrated, wrinkled, or micro-needled.

7. The drug injection device based on an electrochemical reaction according to claim 2, wherein the driving component further comprises a driving cavity covering the electrochemical element, and the driving cavity is located inside the drug storage component;
the driving cavity is used for loading an electrolyte, and the electrolyte undergoes electrochemical reaction under the action of the electrochemical element to enable the driving cavity to deform, and the driving force on the drug solution is generated by the deformation of the driving cavity.

8. The drug injection device based on an electrochemical reaction according to claim 1, wherein the drug injection device based on an electrochemical reaction is an insulin injection system, the insulin injection system comprises a drug injection pump based on electrochemical principle, a first sensor, a sensor circuit module, a pump drive circuit module, and a controller, wherein
the first sensor is attached to the skin of a patient and is used for generating a current signal based on glucose in subcutaneous tissue fluid;
the sensor circuit module is connected to the first sensor, and is used for receiving the current signal and outputting a glucose concentration matched with the current signal through an output end of the sensor circuit module;
the controller is provided with a signal input end and a signal output end, the signal input end is connected to the output end of the sensor circuit module and is used for receiving the glucose concentration, and the signal output end is configured to output a control signal matched with the glucose concentration;
the pump drive circuit module is respectively connected to the signal output end and an electrode of the drug injection pump, and is used to output a driving current or a driving voltage matched with the control signal to the drug injection pump;
the drug injection pump is used for injecting insulin into the patient based on electrochemical reaction under the drive of the driving current or the driving voltage.

9. The drug injection device based on an electrochemical reaction according to claim 8, wherein the drug injection pump comprises a driving component and a drug storage component;
the driving component is arranged inside the drug storage component, and is used to generate a driving force based on an electrochemical reaction principle, the driving force is applied inside the drug storage component, the driving component comprises an electrochemical element which is connected to the outside of the drug storage component via a wire and is used for receiving a preset current; the electrochemical element is used for generating a gas based on the preset current, the gas is used for generating the driving force, and the electrochemical element is an electrode with a nano or micron thickness fabricated by a metal evaporation process, a screen-printing process or a magnetron sputtering process;
the drug storage component is internally loaded with drug solution, and the drug solution is pushed to the outside of the drug storage component along at least one liquid outlet hole on the drug storage component under the driving force, thus administering a drug to a patient through the liquid outlet hole, or administering the drug solution to a patient along an injection mechanism connected to the liquid outlet hole.

10. The drug injection device based on an electrochemical reaction according to claim 8, wherein the first sensor comprises a tubular structure and a plurality of sensor electrodes, the plurality of sensor electrodes are arranged on an outer wall of the tubular structure and are connected to the sensor circuit module.

11. The drug injection device based on an electrochemical reaction according to claim 10, wherein the cross section of the tubular structure is circular, square or polygonal.

12. The drug injection device based on an electrochemical reaction according to claim 1, wherein the drug injection device based on an electrochemical reaction is a closed-loop control system, the closed-loop control system comprises:
an electrochemical micropump, a second sensor, and a control module;
the electrochemical micropump comprises a pump body, the pump body is provided with an accommodation region in which media solution and an electrode layer connected to an inner wall of the pump body are provided, and the pump body is provided with an expansion membrane covering the accommodation region;
the second sensor comprises a substrate, a microneedle array, and an electrode overlying the substrate, the microneedle array is integrally molded with the substrate, and comprises a plurality of hollow microneedles, and each hollow microneedle is internally provided with an injection channel;
the expansion membrane is connected to the substrate of the second sensor, and the tip of the hollow microneedle faces one side away from the expansion membrane;
an input end of the control module is connected to an output end of the second sensor, an output end of the control module is connected to an input end of the electrochemical micropump, the control module is used for receiving an electrical signal output by the second sensor and controlling the turn-on and turn-off of the electrochemical micropump according to the electrical signal.

13. The drug injection device based on an electrochemical reaction according to claim 12, wherein
the control module comprises a first conversion subunit, a control subunit, and a second conversion subunit;
an input end of the first conversion subunit is connected to an output end of the second sensor, an output end of the first conversion subunit is connected to an input end of the control subunit, and the first conversion subunit is used for receiving and converting the electrical signal output by the second sensor;
the control subunit is used for receiving an electrical signal converted by the first conversion subunit and sending a command to the second conversion subunit according to the electrical signal;
an input end of the second conversion subunit is connected to an output end of the control subunit, and an output end of the second conversion subunit is connected to an input end of the electrochemical micropump, and the second conversion subunit is used for receiving and converting the command output by the control subunit, and transmitting the converted command signal to the electrochemical micropump to control the turn-on or turn-off of the electrochemical micropump.

14. The drug injection device based on an electrochemical reaction according to claim 13, wherein
the first conversion subunit is a first signal converter;
the control subunit is a microcontroller;
the second conversion subunit is a second signal converter.

15. The drug injection device based on an electrochemical reaction according to claim 12, wherein
the expansion membrane is made of at least one of polytetrafluoroethylene, polydimethylsiloxane, polyacrylate, silica gel, rubber, latex, polyurethane, parylene, or polyimide.

16. The drug injection device based on an electrochemical reaction according to claim 12, wherein
the electrode layer is made of a hard membrane or a flexible membrane.

17. The drug injection device based on an electrochemical reaction according to claim 12, wherein
the electrode comprises a working electrode and a power supply electrode.

18. The drug injection device based on an electrochemical reaction according to claim 12, wherein
the power supply electrode is a counter electrode;
or, the power supply electrode is a counter electrode or a reference electrode.

19. The drug injection device based on an electrochemical reaction according to claim 12, wherein the drug injection device based on the electrochemical reaction is a closed-loop control system for insulin injection, and the closed-loop control system for insulin injection comprises a closed-loop control system.

20. A fabrication method for a drug injection pump, which is used for fabricating the drug injection pump according to claim 7, wherein the fabrication method includes the steps of:
manufacturing an electrode on a substrate;
bonding a driving cavity to the substrate to completely cover the electrode, and perfusing an electrolyte in the driving cavity; and
forming a drug storage component in the substrate, and enabling the drug storage component to completely encase the driving cavity.
